SEQUENCE LISTING (36 sequences)

SEQ ID NO: 1 - protein, 25 aa, Artificial Sequence
  synthetic
FETLRGDLRILSILRHQNLLKELQD

SEQ ID NO: 2 - protein, 14 aa, Artificial Sequence
  synthetic
FETLRGDLRILSIL

SEQ ID NO: 3 - protein, 10 aa, Artificial Sequence
  synthetic
MLEVESGPPH

SEQ ID NO: 4 - protein, 6 aa, Artificial Sequence
  synthetic
ESGPPH

SEQ ID NO: 5 - protein, 6 aa, Artificial Sequence
  synthetic
ESGPPH

SEQ ID NO: 6 - protein, 8 aa, Artificial Sequence
  synthetic
ETGPSISP

SEQ ID NO: 7 - protein, 9 aa, Artificial Sequence
  synthetic
FPFGPPHWG

SEQ ID NO: 8 - protein, 3 aa, Artificial Sequence
  synthetic
GPD

SEQ ID NO: 9 - protein, 3 aa, Artificial Sequence
  synthetic
GPD

SEQ ID NO: 10 - protein, 3 aa, Artificial Sequence
  synthetic
GPD

SEQ ID NO: 11 - protein, 4 aa, Artificial Sequence
  synthetic
ETGP

SEQ ID NO: 12 - protein, 6 aa, Artificial Sequence
  synthetic
ETFGPD

SEQ ID NO: 13 - protein, 9 aa, Artificial Sequence
  synthetic
TETFGPSID

SEQ ID NO: 14 - protein, 7 aa, Artificial Sequence
  synthetic
ETFGPSI

SEQ ID NO: 15 - protein, 4 aa, Artificial Sequence
  synthetic
GPSI

SEQ ID NO: 16 - protein, 25 aa, Artificial Sequence
  synthetic
FETLRGDERILSILRHQNLLKELQD

SEQ ID NO: 17 - protein, 25 aa, Artificial Sequence
  synthetic
FETLRGEERILSILRHQNLLKELQD

SEQ ID NO: 18 - protein, 15 aa, Artificial Sequence
  synthetic
FETLRGDERILSILR

SEQ ID NO: 19 - protein, 25 aa, Artificial Sequence
  synthetic
  MOD_RES (16)..(16): L-proparglyglycine
  MOD_RES (20)..(20): L-e-azido-norleucine
FETLRGDLRILSILRXQNLXKELQD

SEQ ID NO: 20 - protein, 20 aa, Artificial Sequence
  synthetic
NAVPNLRGDLQVLAQKVART

SEQ ID NO: 21 - DNA, 78 bp, Artificial Sequence
  synthetic
tttgagacactccgaggagatgaacggatcctttccattctgagacatcagaatttactgaaggagctccaagacatg

SEQ ID NO: 22 - DNA, 78 bp, Artificial Sequence
  synthetic
gtcttggagctccttcagtaaattctgatgtctcagaatggaaaggatccgttcatctcctcggagtgtctcaaacat

SEQ ID NO: 23 - protein, 26 aa, Artificial Sequence
  synthetic
  MOD_RES (17)..(17): L-proparglyglycine
  MOD_RES (21)..(21): L-e-azido-norleucine
CFETLRGDLRILSILRXQNLXKELQD

SEQ ID NO: 24 - protein, 25 aa, Artificial Sequence
  synthetic
TTGRRGDLATIHGMNRPFLLLMATP

SEQ ID NO: 25 - protein, 25 aa, Artificial Sequence
  synthetic
DDHGRGDLGRLKKQKDHHNPHLILM

SEQ ID NO: 26 - protein, 25 aa, Artificial Sequence
  synthetic
VPNLRGDLQVLAQKVARTLPTSFNY

SEQ ID NO: 27 - protein, 25 aa, Artificial Sequence
  synthetic
LISRRGDMSSNPAKETFTTGLDAPR

SEQ ID NO: 28 - protein, 25 aa, Artificial Sequence
  synthetic
PQVTRGDVFTMPEDEYTVYDDGEEK

SEQ ID NO: 29 - protein, 18 aa, Artificial Sequence
  synthetic
CDGRCGVRSSSRTPSDKY

SEQ ID NO: 30 - protein, 9 aa, Artificial Sequence
  synthetic
DLSASMDDD

SEQ ID NO: 31 - protein, 21 aa, Artificial Sequence
  synthetic
VKTTPEEIANPCSSIPYFCLP

SEQ ID NO: 32 - protein, 9 aa, Artificial Sequence
  synthetic
ISANIDTPE

SEQ ID NO: 33 - protein, 9 aa, Artificial Sequence
  synthetic
DVSASMHNN

SEQ ID NO: 34 - protein, 21 aa, Artificial Sequence
  synthetic
ISIHPERIHNQCSDYNLDCMP

SEQ ID NO: 35 - protein, 9 aa, Artificial Sequence
  synthetic
ISGNIDTPE

SEQ ID NO: 36 - protein, 26 aa, Artificial Sequence
  synthetic
CFETLRGEERILSILRHQNLLKELQD

(19) United States
(12) Patent Application Publication
(10) Pub. No.: US 2022/0402980 A1
(43) Pub. Date: Dec. 22, 2022

(54) CHROMOGRANIN A-DERIVED PEPTIDES AND USES THEREOF

(71) Applicant: OSPEDALE SAN RAFFAELE S.R.L., Milano (Ml) (IT)

(72) Inventors: Flavio CURNIS, Milano (Ml) (IT); Angelo CORTI, Milano (Ml) (IT); Giovanna MUSCO, Milano (Ml) (IT); Michela GHITTI, Milano (Ml) (IT); Francesca NARDELLI, Milano (Ml) (IT); Alessandro GORI, Vimercate (MB) (IT)

(73) Assignee: OSPEDALE SAN RAFFAELE S.R.L., Milano (Ml) (IT)

(21) Appl. No.: 17/777,228

(22) PCT Filed: Nov. 16, 2020
(86) PCT No.: PCT/EP2020/082257

§ 371 Date: May 16, 2022

(30) Foreign Application Priority Data
Nov. 15, 2019 (EP) 19209484.5

(51) Int. Cl. C07K 14/47; A61K 49/00; A61K 47/64; A61K 51/08

(52) U.S. Cl. CPC C07K 14/47; A61K 49/0056; A61K 47/64; A61K 51/08

(57) ABSTRACT

The present invention refers to chromogranin A-derived peptides that are potent dual ligands for integrins αvβ6 and avβ8, their therapeutic and diagnostic uses and relative compositions.

### TECHNICAL FIELD

The present invention refers to chromogranin A-derived peptides that are potent dual ligands for integrins αvβ6 and αvβ8, their therapeutic and diagnostic uses and relative compositions.

### BACKGROUND ART

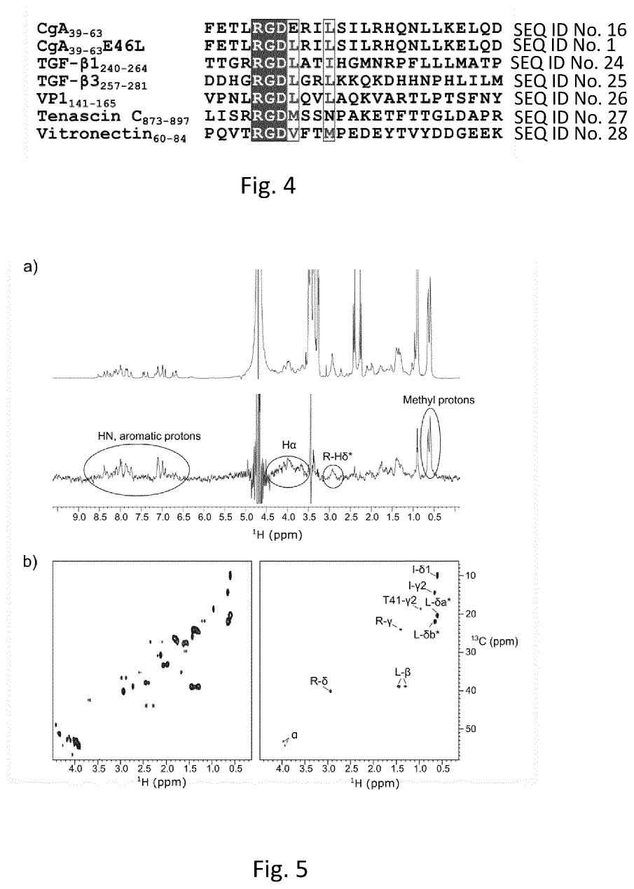

Integrins αvβ6 and αvβ8 are epithelial-specific cell-adhesion receptors, playing a fundamental role in pro-fibrotic cytokine Transforming growth factor beta (TGFβ) activation in fibrosis[1]. They are also highly expressed during tissue remodelling, wound healing, cancer cell migration, invasion and growth, whereby over-expression correlates with poor patient prognosis.[2,3] Hence, targeting of cells highly expressing one or both integrins through high affinity ligands with dual specificity and reduced off-targeting effects may represent a valid, yet poorly explored pharmacological strategy against cancer and/or fibrosis. αvβ6 and αvβ8 are structurally [4] and functionally related [3], albeit αvβ8 [5,6] and its inhibition is far less studied than αvβ6 [7-13].- Both integrins bind to arginine-glycine-aspartate (RGD) containing extracellular matrix proteins, whereby selective recognition occurs through the LXXL/I motif contiguous to the RGD sequence (RGDLXXL/1) [5,14], which folds into one-helical turn upon binding to the receptor, thereafter engaging in specific lipophilic interactions with the hydrophobic pocket of the P6 or 38 subunit [5,15-18]. The inventors have previously shown that human chromogranin A (CgA), a neurosecretory protein involved in cardiovascular system, metabolism, and tumor physiology [19,20]regulation is a natural ligand of αvβ6 [21]. A CgA-derived peptide (residues 39-63) (1) also recognizes αvβ6 with nanomolar affinity and high selectivity (Ki: 15.5±3.2 nM) (Table 3), herewith regulating αvβ6-dependent keratinocyte adhesion, proliferation, and migration [21]. Notably, 1 harbours a degenerate RGDLXXL/I motif, with a glutamate replacing a leucine after the RGD sequence (position D+1, RGDEXXL) (FIG. 4).

Stapled peptides have emerged as an exciting class of therapeutic agents for targeting intracellular protein-protein interactions (PPIs), which have been challenging targets for conventional small molecules and biologics. Verdine G. L., et al., Methods Enzymol. 503, 3-33 (2012); Walensky, L. D,, et al., J. Med Chem. 57, 6275-6288 (2014). They recapitulate the structure and specificity of bioactive a-helices, resist proteolytic degradation in vivo, and, when appropriately designed, gain access to the cytosol and nucleus of mammalian cells. The first cellular application of hydrocarbon-stapled alpha-helices, which were modelled after the BCL-2 homology 3 (BHD) domain of the pro-apoptotic protein BID, revealed their capacity for cellular uptake by an energy-dependent macropinocytotic mechanism, resulting in activation of the apoptotic signaling cascade. Chu, Q., et al., Med. Chem. Commim. 6, 111-1 19 (2015) (clinicaltrials.gov identifier: NCT02264613).

Despite the remarkable promise of stapled peptides as a novel class of therapeutics for targeting previously intractable proteins, designing stapled peptides with consistent cell-permeability remains a major challenge. Many factors including alpha-helicity, positive charge, peptide sequence, and staple composition and placement appear to affect cell uptake propensity. Recently, comprehensive analyses of several hundred stapled peptides in the Verdine and Walensky labs suggest that an optimal hydrophobic, positive charge, and helical content and proper staple placement are the key drivers of cellular uptake, whereas excess hydrophobicity and positive charge can trigger membrane lysis at elevated peptide dosing. See Chu, Q, et al., Med. Chem. Commun. 6, 1 1 1-119 (2015); Nature Chemical Biology. 12, 845-852 (2016). It is clear from these studies that many stapled peptides are either impermeable or poorly permeable to the cell membrane, which limits the application of stapled peptides as therapeutic agents.

Thus, there is a need in the art for peptides with high specificity and affinity for αvβ6 and αvβ8 and for improved stapled peptides having cellular permeability and stability.

### SUMMARY OF THE INVENTION

Combining 2D STD-NMR, computation, biochemical assays and click-chemistry the inventors have identified chromogranin-A derived compounds, such as (4) and (5), that have high affinity and bi-selectivity for αvβ6 and αvβ8 integrins. Further (5) is particularly stable in microsomes. Chromogranin-A derived compounds of the invention are suitable for nanoparticle functionalization and delivery to cancer cells, as potent tools for diagnostic and/or therapeutic applications.

Therefore, the invention provides a peptide comprising an amino acid sequence having at least 65% identity with SEQ ID No. 1 (FETLRGDLRILSILRHQNLLKELQD) or a functional fragment thereof said peptide or functional fragment thereof being in a linear form or in an intramolecular macrocyclic form.

Preferably, the peptide or functional fragment thereof is a ligand of integrins αvβ6 and αvβ8.

Preferably the peptide or functional fragment thereof has a Ki for αvβ6 lower than 2 nM and/or a Ki for αvβ8 lower than 10 nM.

Preferably the peptide or functional fragment thereof comprises FETLRGDLRILSIL (SEQ ID No. 2).

Still preferably the intramolecular macrocyclic form is obtained by a stapling method or is a head-to-tail cyclic form.

Preferably the intramolecular macrocyclic form comprises a triazole-bridged macrocyclic scaffold.

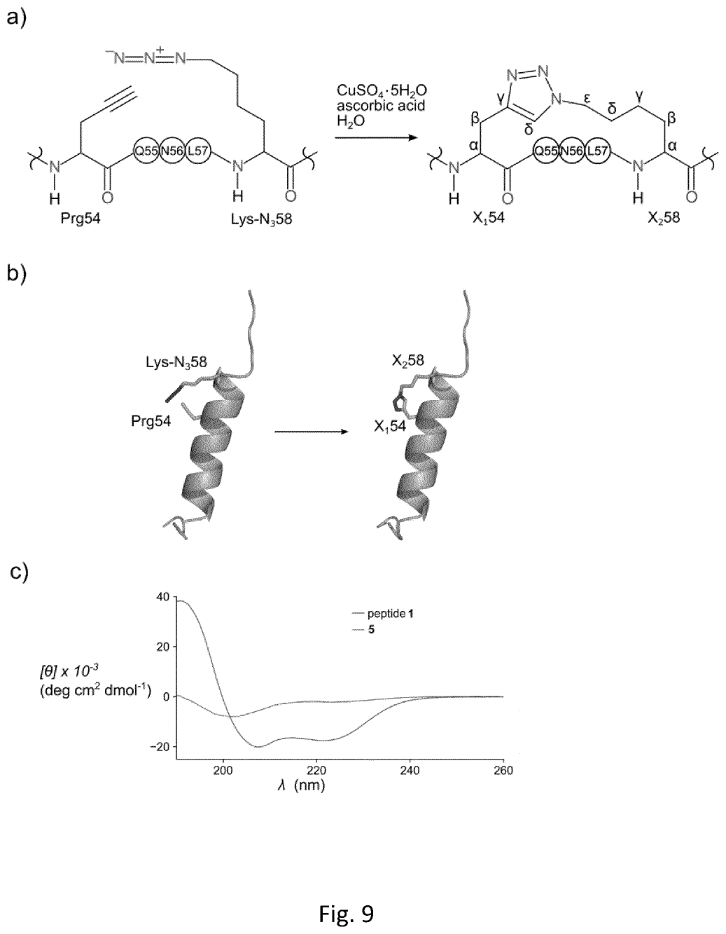

More preferably the triazole-bridged macrocyclic scaffold is present between residues in position 54 (propargylglycine) and 58 (azidolysine) of SEQ ID No. 1 as shown in FIG. 9.

Still preferably the triazole-bridged macrocyclic scaffold is inserted through copper-catalyzed azide-alkyne cycloaddition.

More preferably the intramolecular macrocyclic form has the structure of:

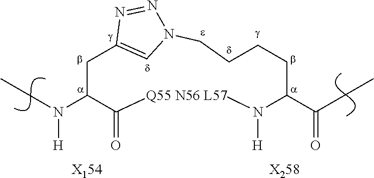

In a preferred embodiment the peptide or functional fragment thereof is coupled or fused with an agent, preferably said agent is an inorganic or organic nanoparticle (e.g. metal nanoparticles, carbon nanoparticles, magnetic nanoparticles, nanocomposites, nanospheres, nanocapsules, nanotubes, liposomes, multilamellar liposomes, micelles, biodegradable/biocompatible nanoparticles, dendrimers, quantum dots, mesoporous silica nanoparticles, polymeric nanoparticles, exosomes and vesicles), therapeutic agents (e.g. cytokines, preferably, but not limited to: tumor necrosis factor (TNF) family members, TNF-related apoptosis inducing ligand (TRAIL), endothelial monocyte activating polypeptide II (EMAP-II), IL12, IFNgamma and IFNalpha, IL18), radioisotopes (e.g. ⁹⁰Y, ¹³¹I, ¹⁷⁷Lu), chemotherapeutic drugs (preferably but not limited: to doxorubicin, melphalan, gemcitabine, taxol, cisplatin, vincristine, or vinorelbine), antibodies and antibody fragments (preferably, but not limited to immune check point blockers, such as anti-PD1 or anti-PDL1 or anti-CTLA4 antibodies, or anti-HER2 antibodies), toxins (e.g. fungal biotoxins, microbial toxins, plant biotoxins, or animal biotoxins that preferably target either DNA or tubulin or other cellular components preferably, but not limited to: duocarmycins, calicheamicins, pyrrolobenzodiazepines, SN-38; MMAE (auristatins monomethyl auristatin E); MMAF (monomethyl auristatin F)), nucleic acids (e.g. antisense oligonucleotides, DNA aptamers, cDNA, and RNA therapeutics (micro RNAs, short interfering RNAs, ribozymes, RNA decoys and circular RNAs)), diagnostic agents for radioimaging (PET, CT, and SPECT), fluorescence and photoacoustic imaging (e.g. radioisotopes fluorescent dyes or nanoparticles preferably, but not limited to: ¹⁸F, ⁶⁷Ga, ⁶⁸Ga, ^{81m}Kr, ⁸²Rb, ¹³N, ^{99m}Tc, ¹¹¹In, ¹²³I, ¹³³Xe, ²⁰¹Tl, fluoresceins, rhodamines, bodipys, indocyanines, porphyrines andphthalocyanines, IRDye ICG, methylene blue, omocyanine and quantum dots), a dye (such as NOTA), a contrasting agents for MRI and Contrast-enhanced ultrasound (CEUS) (e.g, gadolinium-based compounds, superparamagnetic iron oxide (SPIO) and ultrasmall superparamagnetic iron oxide (USPIO) compounds, and microbubbles) or cellular components (CAR-T cells, lymphocytes, NK cells, macrophages and dendric cells).

The invention also provides a composition comprising the peptide or functional fragment thereof according to any one of previous claim and suitable carriers.

Preferably the composition further comprises an agent, preferably said agent is a nanoparticle, a therapeutic agent, a contrasting agent or a cellular component.

The peptide or functional fragment thereof according to the invention or the composition of the invention is for use as a diagnostic or therapeutic agent, preferably for use as a diagnostic imaging agent.

Preferably for use in detecting a tumor, preferably the tumor overexpresses αvβ6 and αvβ8 integrins, preferably the tumor is oral or skin squamous cell carcinoma, head and neck, pancreatic, ovarian, lung, cervix, colorectal, gastric, prostatic and breast cancer, melanomas and brain tumors (e.g. glioblastoma and/or astrocytoma).

Preferably for use in the treatment of cancer or fibrosis, preferably the cancer expresses high levels of integrins αvβ6 and αvβ8, preferably the cancer is oral or skin squamous cell carcinoma, head and neck, pancreatic, ovarian, lung, cervix, colorectal and breast cancer, brain tumors (e.g. glioblastoma and astrocytoma).

Preferably the peptide or a fragment of the peptide comprises the sequence FETLRGDLRILSIL (SEQ ID No. 2).

The present invention is illustrated by means of non-limiting examples in reference to the following figures.

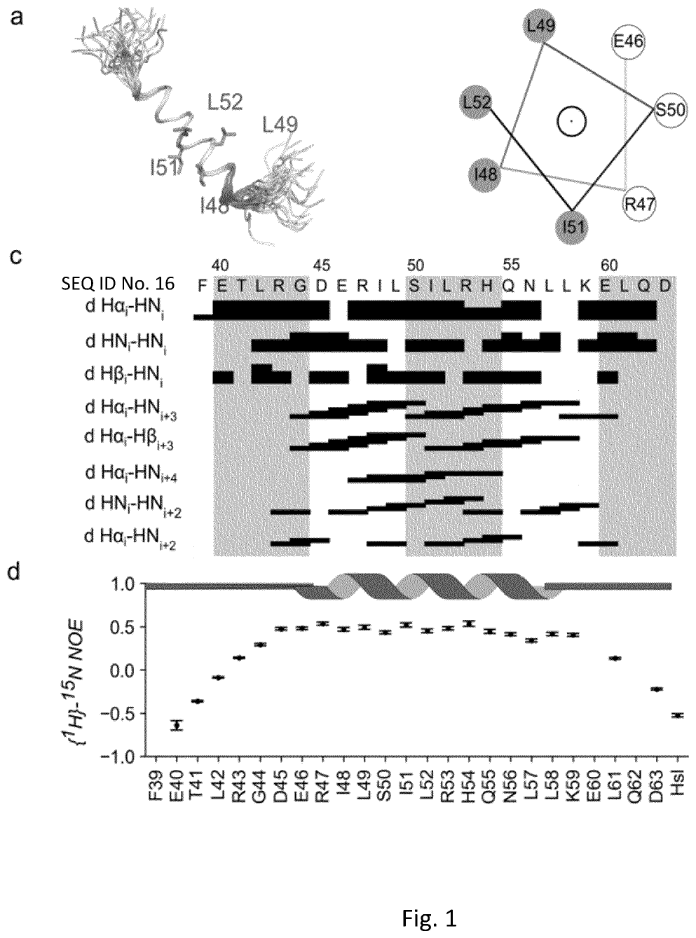

FIG. 1. Solution structure of peptide 1. a) Representation of the 15 lowest energy NMR structures (pdb code: 6R2X) aligned on E46-N56 backbone atoms with the RGD motif in orange and 148, L49, 151 and L52 in green. b) Helical wheel projection of residue E46-L52 with hydrophobic residues in green. c) Scheme of medium and short NOEs (Nuclear Overhauser Effects) relevant for secondary structure identification. Height of the boxes is proportional to NOE intensities. d) Sequence specific backbone heteronuclear {¹H}-¹⁵N NOEs with elements of secondary structure indicated on the top.

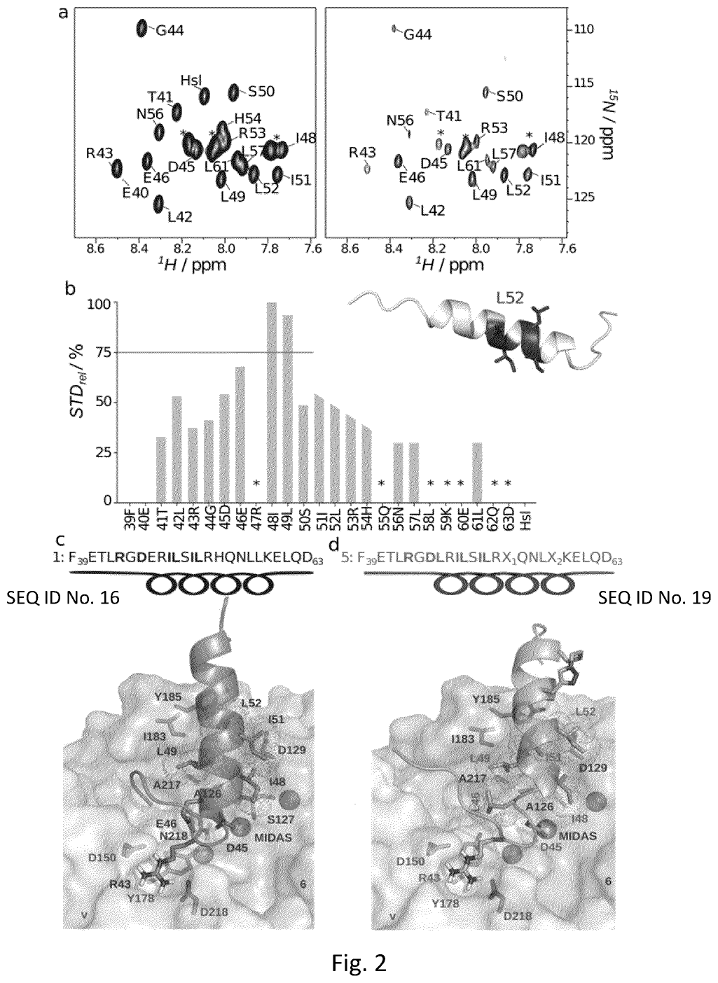

FIG. 2. Interaction of αvβ6 with peptides 1 and 5. a) 2D-STD-¹H-¹⁵N-HSQC experiment performed on ¹⁵N labelled peptide 1 (0.5 mM) in the presence of recombinant extracellular αvβ6 (4 μM), off-resonance (left) and difference spectra (right); asterisk indicates overlapped signals; Hsl (Homoserine lactone). b) Residue-specific STD %, as defined below; asterisks indicate overlapping signals. Residues with STD % >75% are mapped on the 3D structure. c) HADDOCK model of 1 (left) and d) 5 (right) in complex with αvβ6. Ligand and receptor residues involved in the interaction, E46 in 1 and the triazole-containing stapled residues in 5 are shown in sticks. Sequence and secondary structure of peptide 1 and peptide 5 are shown on the top. Interacting residues are highlighted in bold.

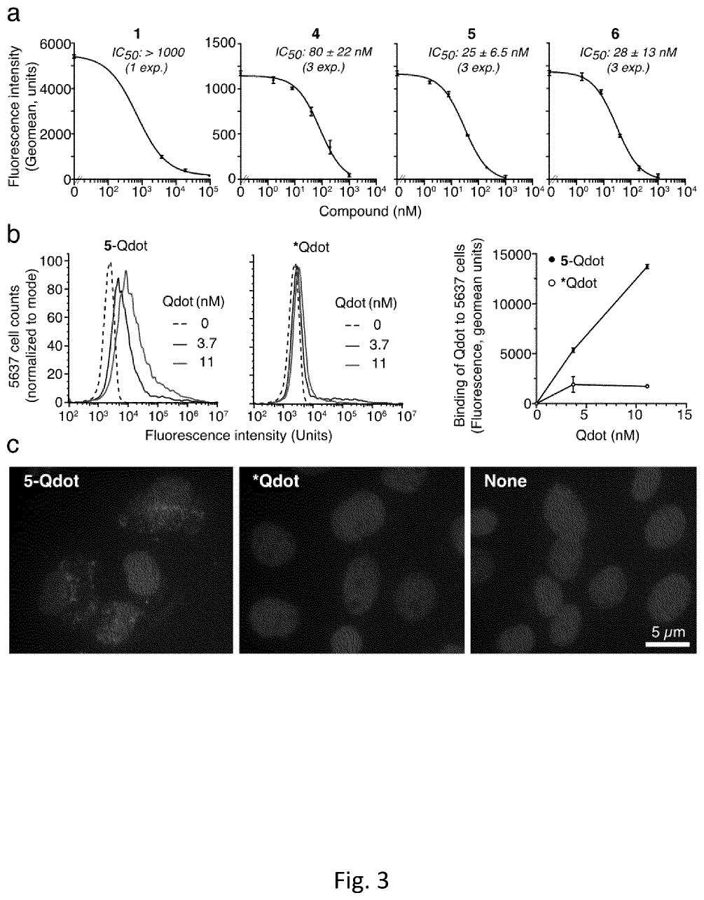

FIG. 3. Binding of CgA-derived peptides to human bladder cancer 5637 cells. a) Effect of 1, 4, 5 and 6 on the binding of anti-αvβ6 mAb 10D5 to 5637 cells. Antibody binding quantification as determined by flow cytometry analysis (FACS) (see also FIG. 11A). Compounds were mixed with mAb 10D5 and added to cells; mAb binding was detected by FACS and inhibitory concentration (IC₅₀, mean±SEM) was determined. Each point is in duplicate. b) Binding of 5-Qdot or *Qdot (control) to 5637 cells as measured by FACS. Representative FACS (left and middle) and quantification of Qdot binding (right) (circles: mean±SD of duplicates). c) Representative fluorescence bioimaging of 5637 cells incubated with 5-Qdot, *Qdot or diluent. Magnification 40×; red, Qdot; blue, nuclear staining with DAPI.

FIG. 4. Multiple Sequence alignment of human CgA with αvβ6 interacting proteins. Alignment of residues 42-53 of human CgA (Uniprot: P10645) and E46L mutant with TGF-P1 (Uniprot: P01137, residues 243-254), TGF-P3 (Uniprot: P10600, residues 260-271), VP1 coat protein of FMDV (Uniprot: B2MZQ8, residues 144-155), tenascin C (Uniprot: P24821, 876-887), vitronectin (Uniprot: P04004, residues 63-74). The alignment was performed with ClustalX[22] on residue 42-53 of CgA and plotted with ESPript3.0.[23] Completely conserved, highly conserved and highly homologous residues are highlighted with a red background, colored in red or boxed, respectively.

FIG. 5. STD experiments of peptide 1 in the presence of recombinant human αvβ6. a) ¹H 1D-STD experiment (lower panel) and corresponding off-resonance spectrum (upper panel) performed on peptide 1 (0.3 mM) in the presence of recombinant extracellular αvβ6 (1.3 μM). b) ¹H-¹³C-HSQC reference spectrum (left) and 2D-STD-1H-¹³C-HSQC spectrum (right) performed on ¹³C/¹⁵N recombinant peptide 1 (0.5 mM) and recombinant extracellular αvβ6 (4 μM). Detectable groups of signals are labelled.

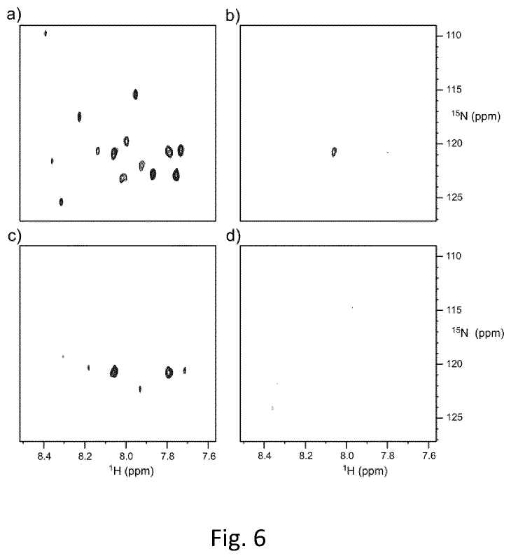

FIG. 6. 2D-STD-¹H-¹⁵N-HSQC spectra of peptide 1. Experiments on 1⁵N labelled peptide 1 (0.5 mM) a) in the presence of recombinant extracellular αvβ6 (4 μM); b) in the presence of bovine serum albumin (4 μM), c) 1 in the presence of recombinant extracellular αvβ6 (4 μM previously treated with 20 mM of EDTA d16, and d) 1 alone.

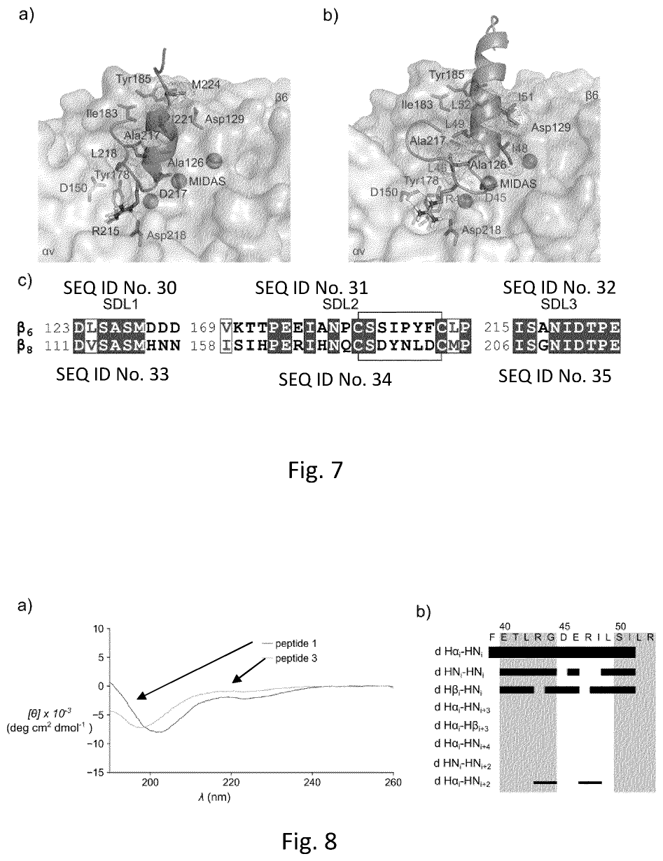

FIG. 7. Structural comparison between TGF-s1 and 4/αvβ6 binding mode and alignement of SDL sequences of $6 and $8. a) Crystal structure of αvβ6 together with TGF-P1 (PDB:5FFO)[16]. b) HADDOCK model of peptide 4/αvβ6 interaction; TGF-1 (magenta) from residue F210 to P227 and peptide 4 (orange) are shown in cartoon representation. av and 36 subunits are represented as pale cyan and green surfaces, respectively, with metal ions shown as spheres. Ligand residues side chains involved in the interaction are shown in sticks and labeled with one-letter code, with side chains of hydrophobic residues highlighted with dots; receptor interacting residues are shown in sticks and labeled with three-letter code; electrostatic interactions are represented with dashed lines. c) Sequence alignment of SDL1, 2, and 3 of 36 and 38 was performed with ClustalX [22] and plotted with ESPript3.0.[23] Completely conserved, highly conserved and highly homologous residues are highlighted with a red background, colored in red or boxed, respectively.

FIG. 8. Circular Dichroism and NMR analysis of 3. a) Overlay of CD spectra of peptide 1 (red) and 3 (orange) (30 lpM), in phosphate buffer 20 mM, NaF 100 mM, pH 6.5, T=280K. b) Schematic representation of medium and short NOE contacts identified in 3. The height of the box is proportional to the NOEs intensities.

FIG. 9. Effect of stapling on the conformation of 1. a) Schematic representation of the “click” reaction used to obtain 5; b) cartoon representation of the precursor and product, with propargylglycine and azidolysine in position 54 and 58 on the left and triazole bridge on the right shown as sticks. c) CD spectra of peptides 1 (red) and 5 (purple) (30 μM), in phosphate buffer 20 mM, NaF 100 mM, pH 6.5, T=280K.

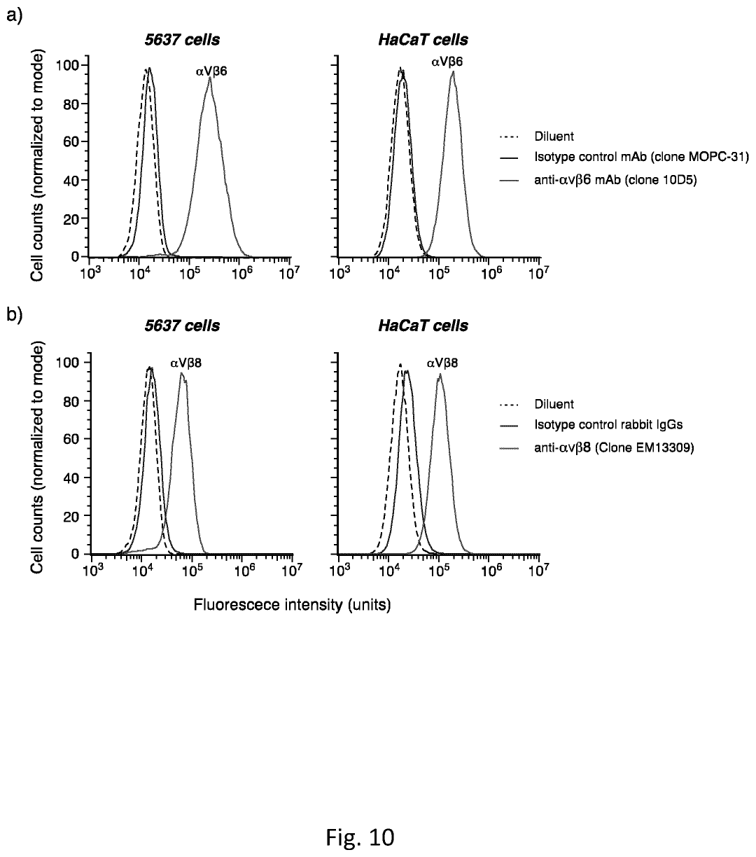

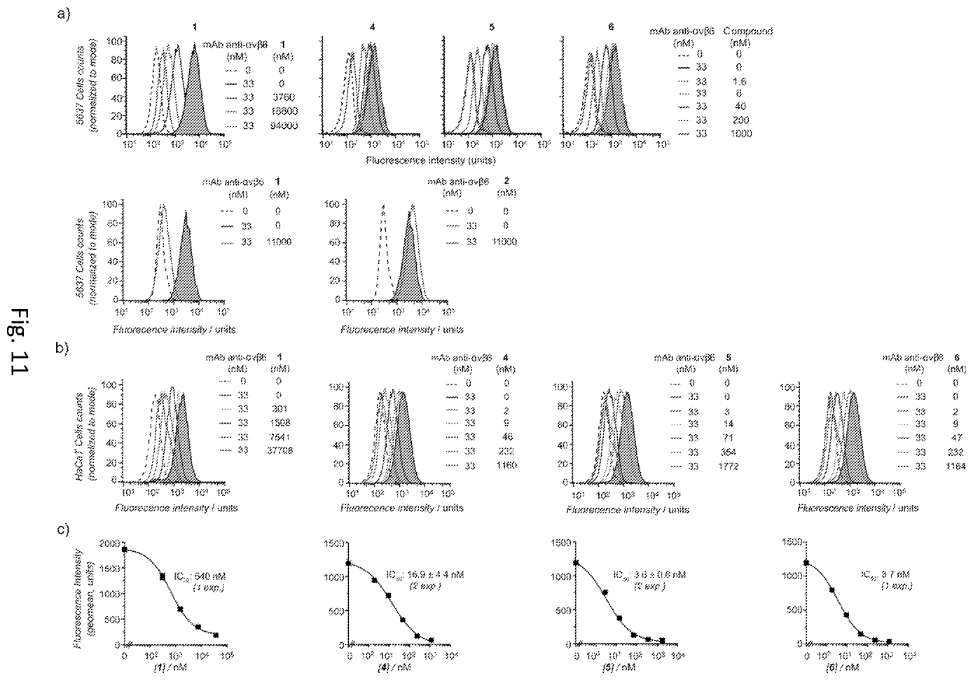

FIG. 10. αvβ6 and avs8 integrin expression on human bladder carcinoma 5637 cells and human skin keratinocytes (HaCaT). Representative flow cytometry analysis of the expression of αvβ6 (a) and αvβ8 integrin (b) as detected by FACS analysis using an anti-αvβ6 mAb (clone 10D5, 5 μg/ml) and an anti-αvβ8 antibody (clone EM13309, 1 μg/ml), followed by a goat anti-mouse or an anti-rabbit Alexa Fluor 488-labeled secondary antibodies (5 μg/ml), respectively. Binding of isotype control antibodies is also shown.

FIG. 11. Effect of peptides 1, 2, 4, 5 and 6 on the binding of anti-αvβ6 mAb 10D5 to human bladder carcinoma 5637 cells and human skin keratinocytes (HaCaT). a) Representative flow cytometry analysis showing the effect of peptide 1, 2, 4, 5 and 6 on the binding of mAb 10D5 to αvβ6 positive 5637 cells. Compounds were mixed with mAb 10D5 and added to cells; mAb binding was detected by FACS. Quantification of mAb binding is reported in FIG. 3A. b) Representative flow cytometry analysis and c) quantification of antibody binding showing the effect of 1, 4, 5 and 6 on the binding of mAb 10D5 to αvβ6 positive HaCaT cells. Squares show mean±SD of duplicates. Inhibitory concentration 50 (ICso, mean±SD) of the indicated number of independent experiments is shown.

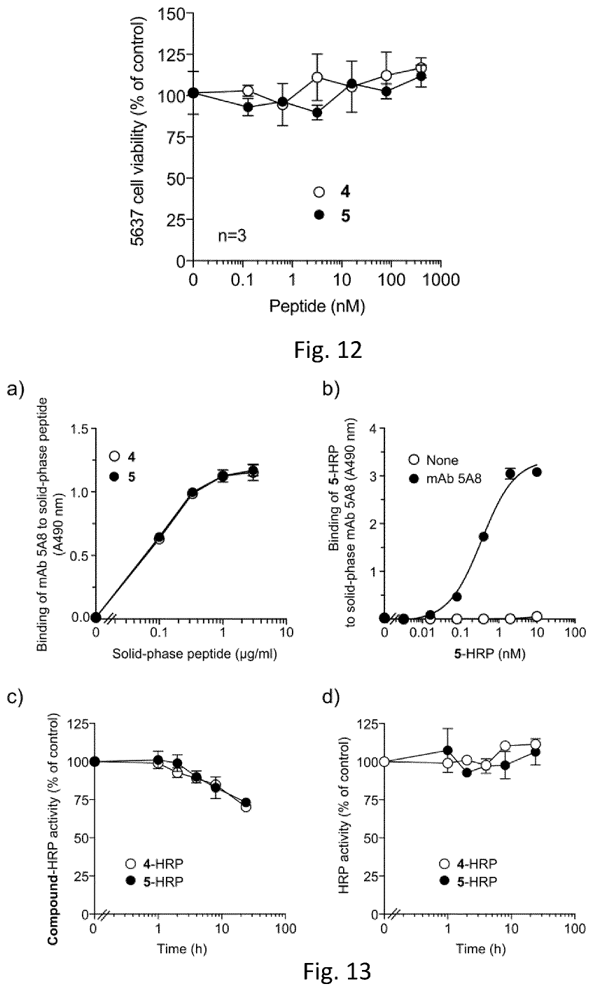

FIG. 12. Effect of peptides 4 and 5 on human bladder carcinoma 5637 cell viability. 5637 cells were seeded in a 96-well microtiterplate (20,000 cell/well) and cultured for 16 h at 37° C., 5% CO₂. The day after the indicated doses of peptide 4 and 5 were added to the cells and left to incubate for additionally 48 h at 37° C., 5% CO₂. Cell viability was assessed using the PrestoBlue* cell viability reagent (ThermoFisher) according to the manufacturer's instructions. Viability of the treated cells was normalized to that of untreated cells and is reported as a percentage (mean±SE of triplicate wells).

FIG. 13. Stability of peptides 4-HRP and 5-HRP in human serum as determined by ELISA. a) Experimental set up of ELISA assays to monitor the stability of 4 and 5 in human serum. Binding of mAb 5A8 (anti-CgAs4.s₇) to microtiterplates coated with peptide 4 or 5. The binding of mAb 5A8 was detected using a peroxidase-labeled goat-anti-mouse antibody and o-phenylendiamine as a chromogenic substrate. The results show that peptide stapling does not impair mAb 5A8 binding. b) Peptide 5-horseradish peroxidase conjugate (5-HRP) assay dose-response curve. Binding of 5-HRP at various concentrations to a microtiterplate coated with or without mAb 5A8 (5 μg/ml) is shown. Each point represents mean±SEM of quadruplicates. c) 4- or 5-horseradish peroxidase conjugates (4-HRP and 5-HRP, respectively) were incubated in human serum at 37° C., collected at different times (0, 1, 2, 4, 8 and 24 h) and added to microtiterplates pre-coated with mAb 5A8 (5 μg/ml). Compound-peroxidase conjugate bound to the plate was determined using the o-phenylendiamine chromogenic substrate of HRP (left panel). d) In parallel, the effect of serum on the peroxidase activity (HRP) of the conjugate was also checked by measuring the enzyme activity using the same chromogenic substrate. Each point represents mean±SEM of quadruplicates.

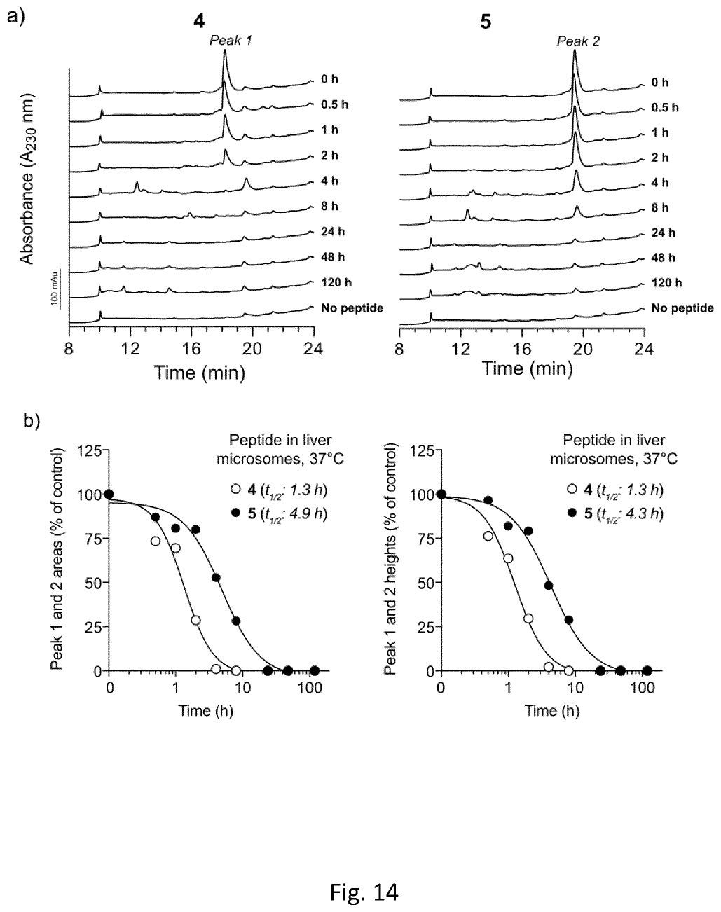

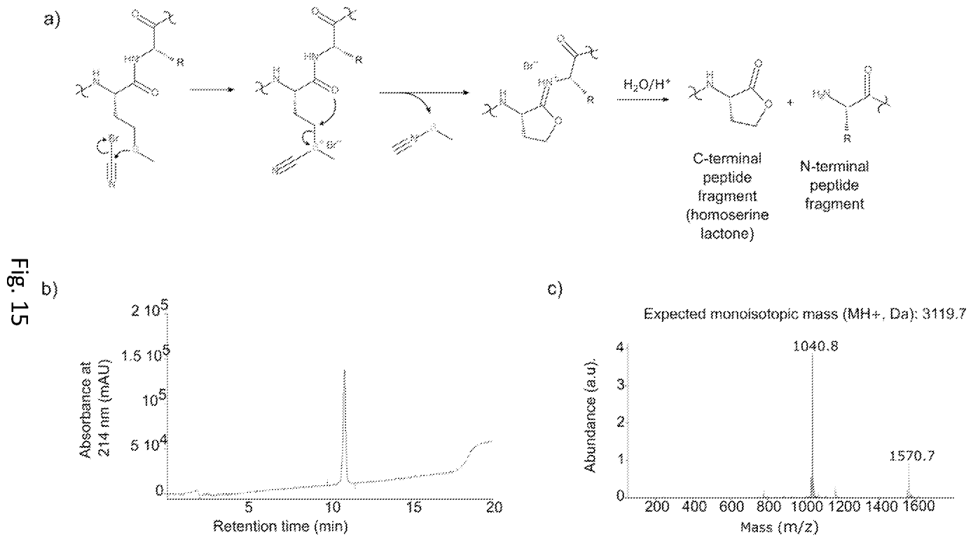

FIG. 14. Stability of peptides 4 and 5 in murine liver microsomes as determined by RP-HPLC. a) RP-HPLC of 4 and 5 after incubation at 37° C. in murine liver microsomes. Peak 1 corresponds to 4 and 5. The peptides were added to murine liver microsomes (454 - μg/ml, final concentration) and incubated for the indicated time, diluted with an equal volume of 90% acetonitrile containing 0.1% TFA and analyzed onto a LiChrospher C18 column (16 ag). No peptide indicates liver microsome aliquot without the peptide. b) Quantification of Peak 1 area (left panel) and height (right panel) of the indicated peptides. The corresponding half-life is also shown.

FIG. 15. Reaction mechanism and purification of recombinant peptide 1. a) Reaction mechanism of the cleavage of methionine-containing peptide with cyanogen bromide. The product of the reaction is a homoserine lactone C-terminal residue. b) Analytical RP-HPLC and c) electro-spray mass spectrometry (ESI-MS) analysis of recombinant peptide 1. ESI-MS was performed using a Bruker Esquire 3000+instrument equipped with an electro-spray ionization source and quadrupole ion trap detector. The mass of the peptide including the lactone [M+H]* is 3119.7 Da and the peaks at 1040.8 Da and 1570.7 Da correspond to [M+3H]³+ and to [M+H+Na]²+, respectively.

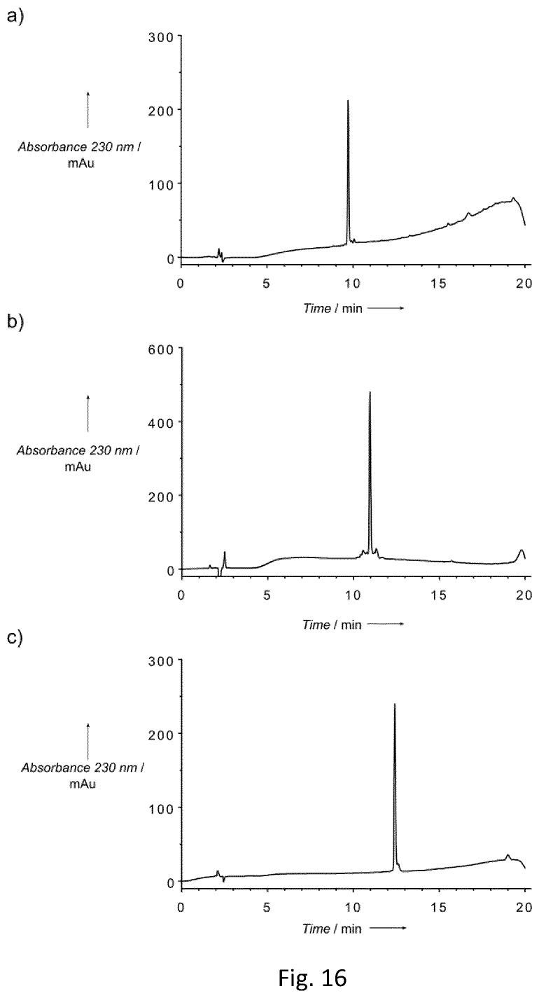

FIG. 16. Analytical RP-HPLC. RP-HPLC of a) 3, b) 4, and c) 5 was carried out on a Shim-pack GWS C18 (5 pm, 4.6×150 mm) using a Shimadzu Prominence HPLC.

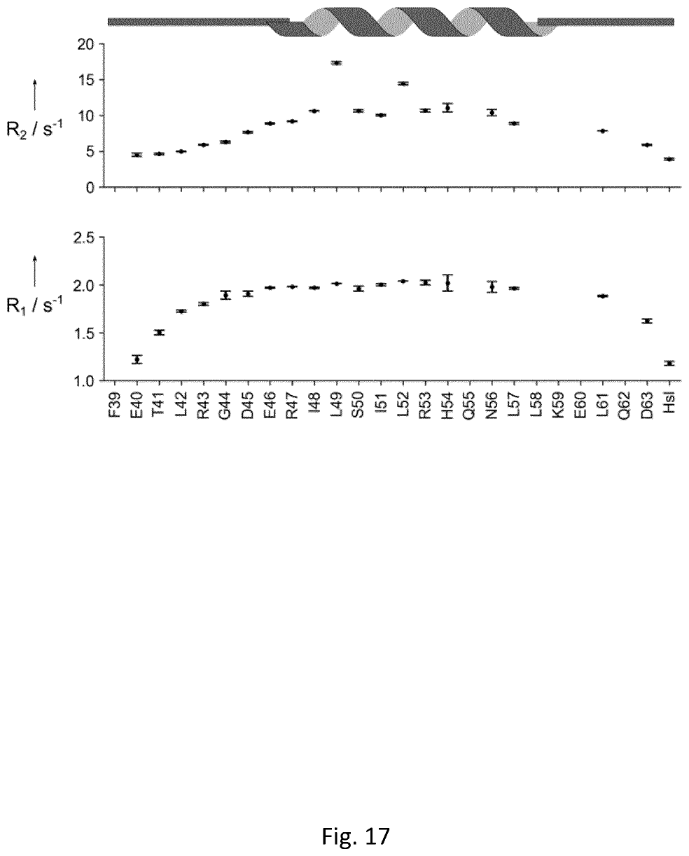

FIG. 17. ¹⁵N Relaxation analysis. ¹⁵N R₁ (bottom) and R₂ (top) relaxation rates measured for recombinant peptide 1; elements of secondary structure are indicated on the top of the figure.

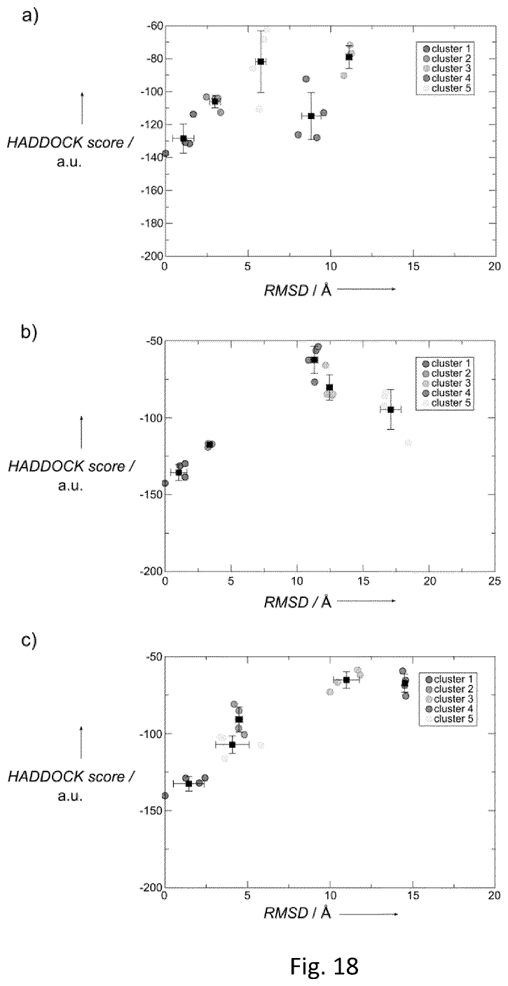

FIG. 18. HADDOCK score of the clusters as a function of their RMSD from the lowest energy structure. Graphs represent HADDOCK score vs RMSD from the lowest energy complex structures in terms of HADDOCK score (a.u.) for the clustered decoy poses of: a) αvβ6/1, b) αvβ6/4, and c) αvβ6/5.

Circles correspond to the best four structures of each cluster; black squares correspond to the cluster averages with the standard deviation indicated by bars. The first best 5 clusters in terms of HADDOCK score are represented.

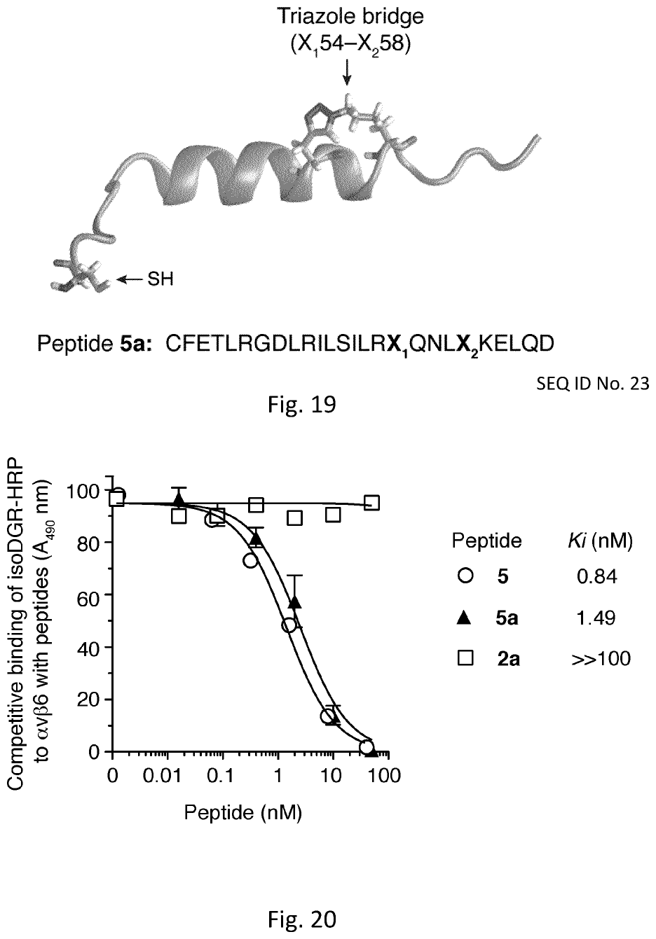

FIG. 19. Cartoon representation of peptide 5a.

Aminoacid sequence of 5a and the triazole bridge obtained by click chemistry reaction between propargylglycine (X1-54) and azidolysine (X2-58) of CgA38-63-derived peptide, are shown.

The N-terminal sulfhydryl of cysteine in position 38 has been used for chemical coupling of 5a.

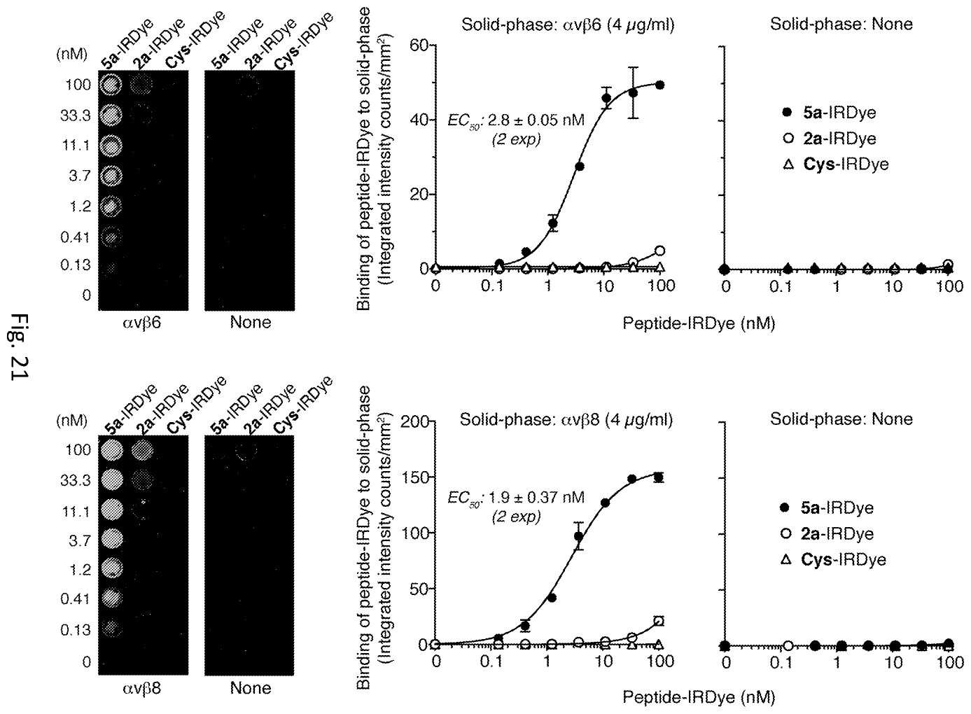

FIG. 20. Competitive binding of isoDGR-peroxidase conjugate with peptide 5, 5a and 2a to αvβ6-coated microtiter plates.

The competitive binding assay was performed as previously described (1), using isoDGR (a mimetic of RGD) labelled with peroxidase as a probe for the integrin binding site.

A representative experiment is shown with the resulting Ki values. Mean ±SE of two technical replicates.

FIG. 21. Binding of peptide-IRDye conjugates to αvβ6- or avs8-coated microtiter plates.

Binding of 5a-IRDye and 2a-IRDye to microtiterplates coated with or without αvβ6 or αvβ8 as indicated in each panel. Various amounts of conjugates were added to microtiterplates and incubated for 1 h at room temperature (see Experimental section). After washing, bound fluorescence was measured using an Odyssey CLx (LI-COR) scanner. Representative images of the scanned plate and quantification of the binding are reported. Mean ±SE of triplicates. Effective concentration 50 (EC50, mean±SD) of the indicated number of independent experiments is shown.

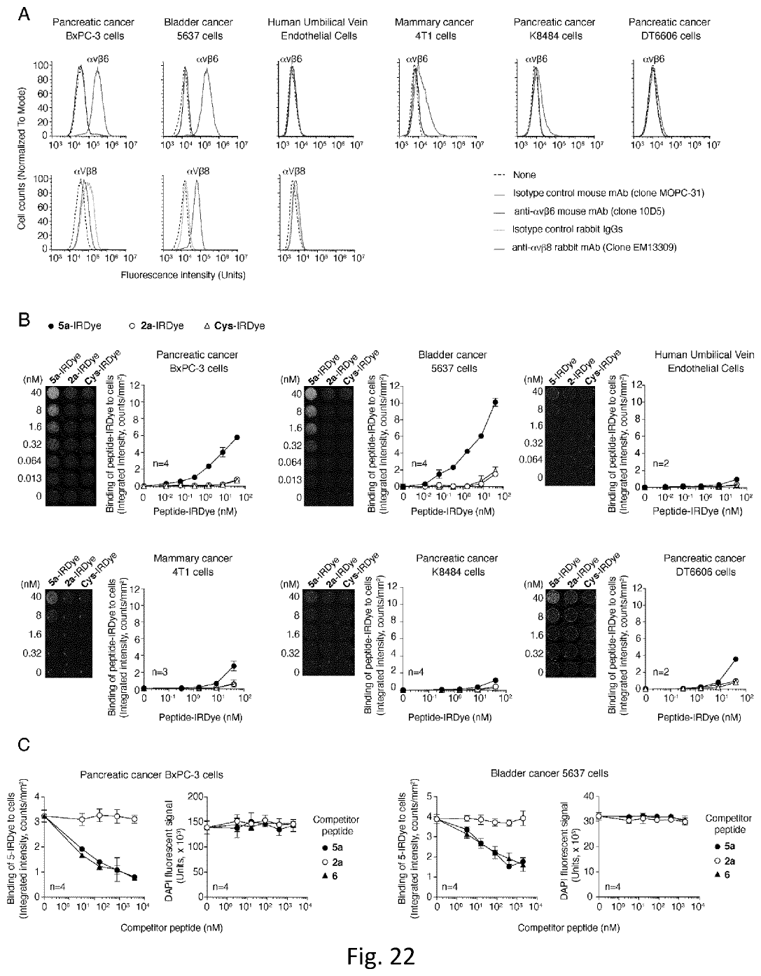

FIG. 22. Binding of 5a-IRDye, 2a-IRDye and Cys-IRDye to BxPC-3, 5637, HUVEC, 4T1, K8484 and DT6606 cells.

A) Expression of αvβ6 and αvβ8 by the various cell lines, as evaluated by a FACS analysis using the indicated monoclonal antibodies, followed by AlexaFluor 488-goat anti-mouse or anti-rabbit IgG polyclonal antibody.

B) Binding of the indicated peptide-IRDye conjugates to the various cell lines. Various amounts of conjugates were added to cell monolayers (grown in 96-well microtiter plates) and incubated for 1 h at 37° C., 5% CO2 (see Experimental section). After washing, bound fluorescence was measured using an Odyssey CLx (LI-COR) scanner. A representative image of the scanned plate and binding quantification are reported. Mean ±SE of quadruplicate wells.

C) Effect of unlabeled peptide 5a, 2a and 6 on the binding of 5a-IRDye to BxPC-3 and 5637 cells.

Various amounts of unlabeled peptides were mixed 5a-IRDye (4 nM) and added to the cells. After 1 h, the plates were washed, and the bound fluorescence was quantified as described above (left panels).

Then the cells were stained with DAPI (a nuclear staining) to quantify the total cells: the bound fluorescence was measured using a fluorescence plate reader (right panels). Mean ±SE of quadruplicate wells.

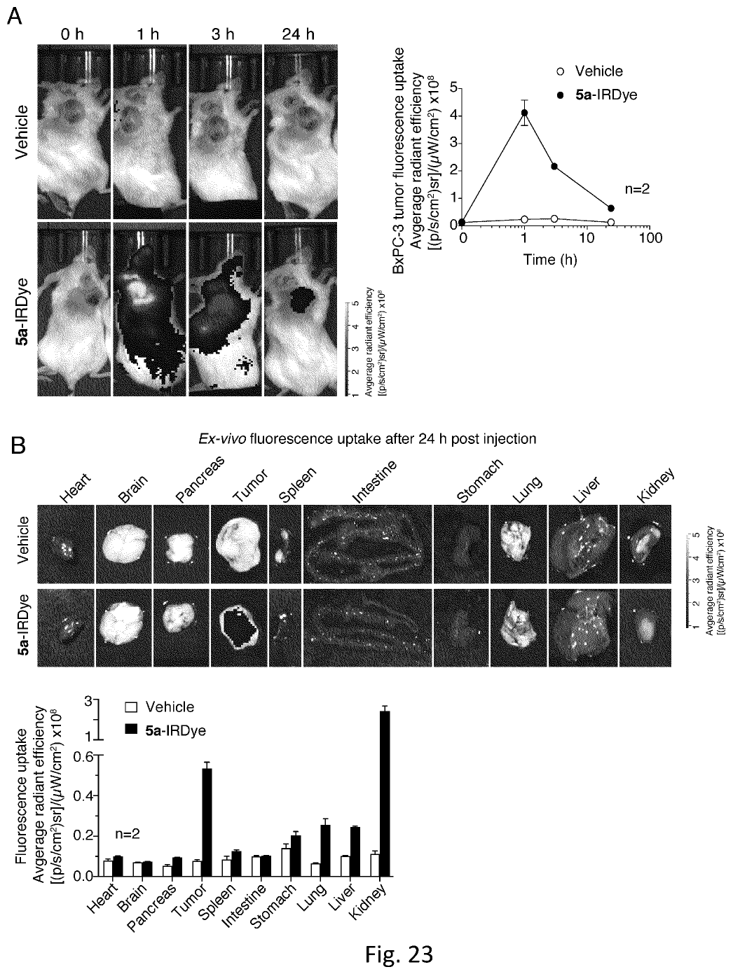

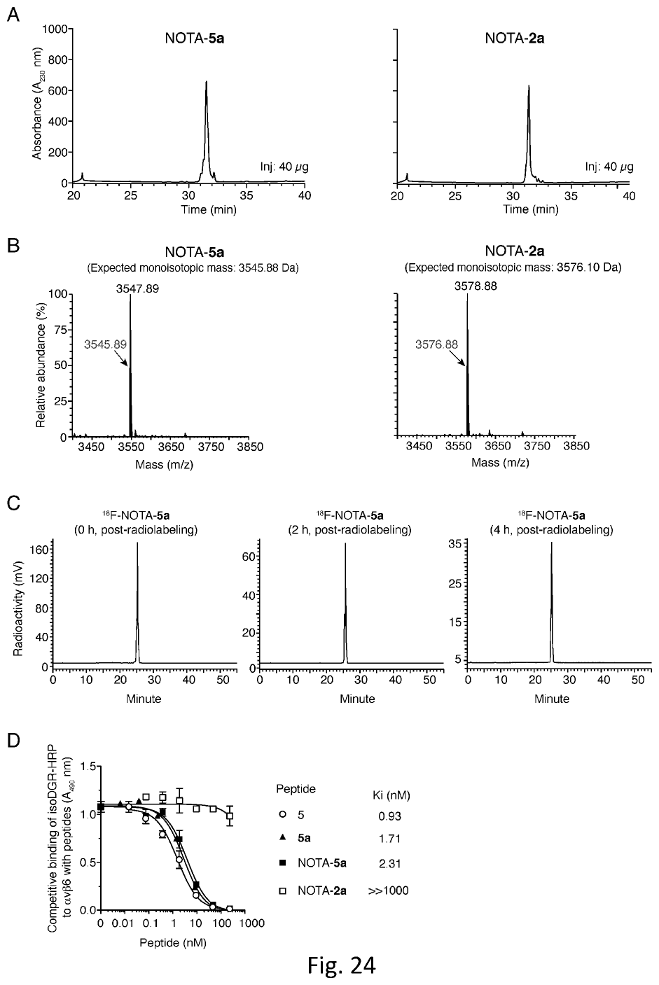

FIG. 23. Tumor uptake and biodistribution of 5a-IRDye in mice bearing subcutaneous BxPC-3 tumors.

Eight-weeks old NGS mice were challenged with BxPC-3 cells on the right shoulder. Thirty-five days later mice were treated with 5 μg of 5a-IRDye or with diluent (vehicle) and subjected to optical imaging using an IVIS SpectrumCT after 1, 3 and 24 h. Animals treated with vehicle served as a reference for the quantification of autofluorescence in the near infrared region.

A) Representative image and quantification of the uptake of the 5a-IRDye in BxPC-3 tumors at the indicated time points (mean±SD of 2 mice per group).
B) Representative images and quantification of the 5a-IRDye uptake (24 h post-injection) by the indicated organs (mean±SD, of 2 mice per group). A and B panels show pseudocolor fluorescence superimposed on a white light image.

FIG. 24. Biochemical characterization of NOTA-5a and NOTA-2a conjugates.

A) Reverse-phase HPLC analysis of NOTA-5a and NOTA-2a conjugates using a LUNA C18 column.

B) Mass spectrometry analysis (LTQ-XL Orbitrap) of purified NOTA-5a and NOTA-2a conjugates. The expected monoisotopic masses are shown.

C) Stability of NOTA-5a after labelling with 18F, as determined by reverse-phase HPLC analysis using an ACE C18 column.

D) Competitive binding of isoDGR-HRP to αvβ6 coated-microtiter plates with peptides and NOTA-peptide conjugates, as measured by competitive ELISA. A representative experiment is shown with the resulting Ki values. Mean ±SE of two technical replicates.

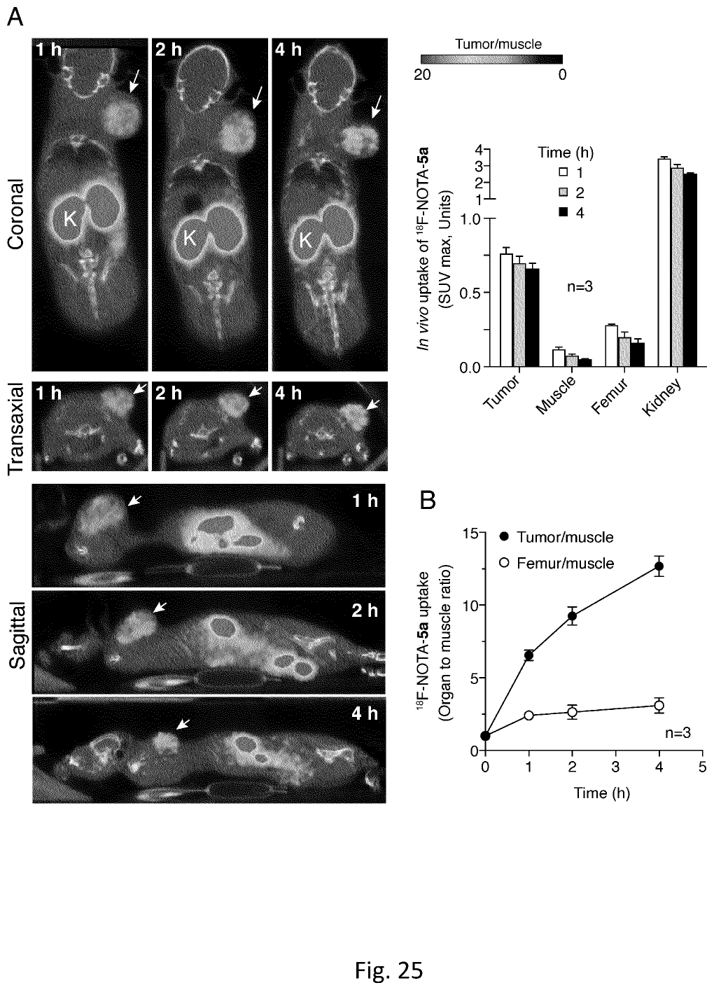

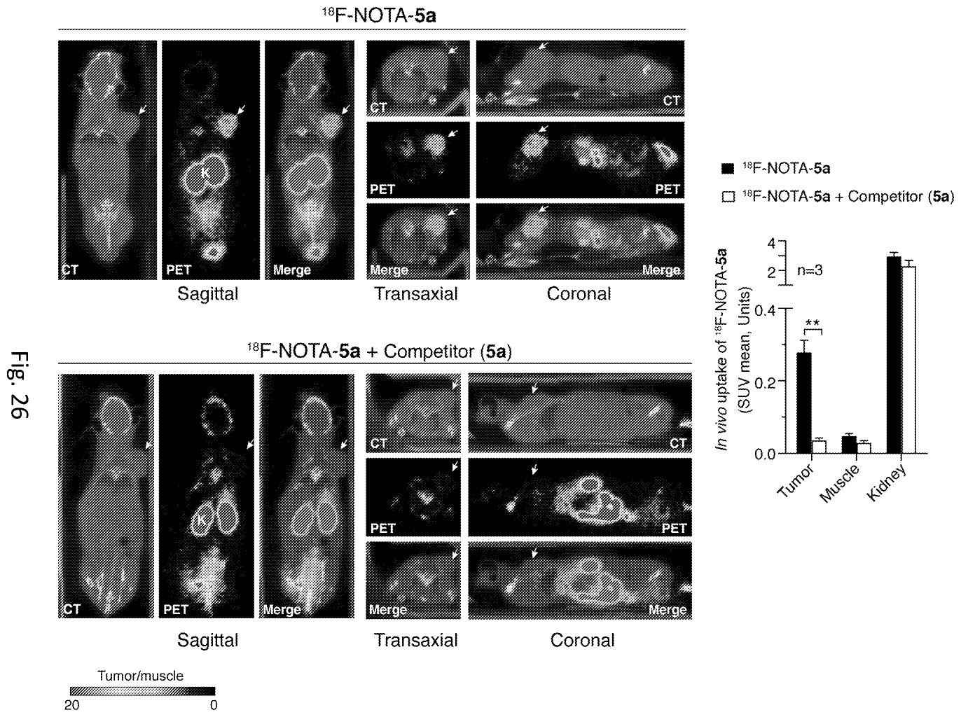

FIG. 25. PET/TC assessment of 18F-NOTA-5a uptake by subcutaneous BxPC-3 tumors.

Mice bearing subcutaneous BxPC-3 tumors, implanted in the right shoulder, were intravenously injected with 18F-NOTA-5a (˜ 4 MBq/mouse) and subjected to whole body PET/CT scan at the indicated times. A) Representative coronal, transaxial and sagittal images and quantification (mean±SE) of the standardized uptake maximum value (SUV max) of radiotracer in the indicated tissues of 3 mice. The large amount of radioactivity in the kidneys (K) is likely related to renal excretion. Arrows, BxPC-3 tumor. B) Kinetics of the uptake of 18F-NOTA-5a in tumor or femur expressed as tumor-to-muscle or femur-to-muscle ratio. Ratio values are presented as mean±SE of 3 mice.

FIG. 26. Competition of 18F-NOTA-5a uptake by unlabeled peptide 5a in the subcutaneous BxPC-3 tumor model.

Mice bearing subcutaneous BxPC-3 tumors, implanted in the right shoulder, were intravenously injected with or without an excess 5a peptide (400 μg, Competitor) followed 10 min later by 18F-NOTA-5a (˜ 3 MBq/animal). After 2 h the mice were subjected to a whole-body PET/CT scan.

Representative coronal, transaxial and sagittal whole-body TC, PET and PET/TC images (merge) and quantification of the standardized uptake mean value (SUV mean) of radiotracer in the indicated organs. SUV mean values are presented as mean±SE of 3 mice.

Arrow, BxPC-3 tumor. **, P<0.01 by two-tail t-test.

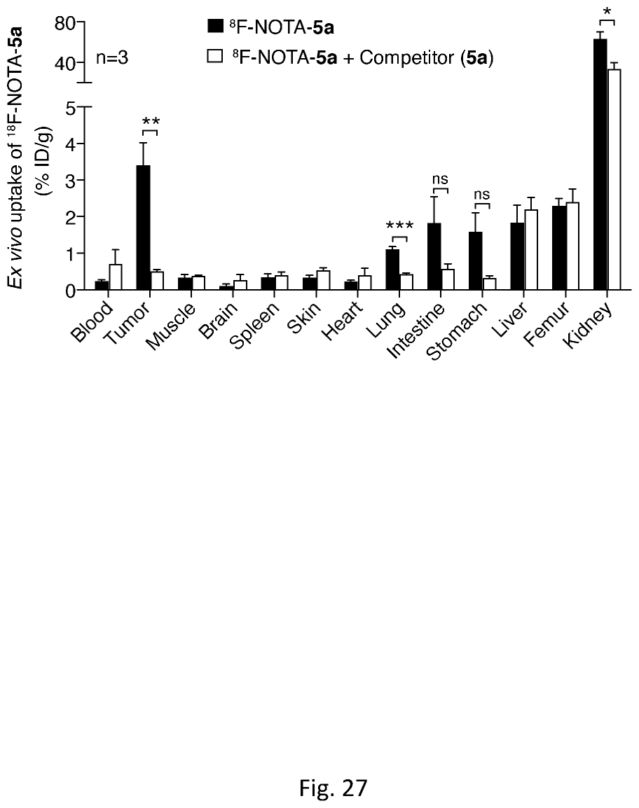

FIG. 27. Biodistribution of 18F-NOTA-5a in mice bearing subcutaneous BxPC-3 tumors.

Mice bearing subcutaneous BxPC-3 tumors, implanted in the right shoulder, were intravenously injected with or without an excess 5a peptide (400 μg, Competitor) followed 10 min later by 18F-NOTA-5a (˜ 3 MBq/animal). Two hours later, the mice were sacrificed. Then, tumors and the indicated organs were excised and analyzed with a gamma-counter for determining the uptake of radiotracer.

Bars: mean±SD (n=3 mice). *, P<0.05, **, P<0.01 and ***P<0.0001 by two-tail t-test.

### DETAILED DESCRIPTION OF THE INVENTION

When describing the present invention, all terms not defined herein have their common art-recognized meanings. Any term or expression not expressly defined herein shall have its commonly accepted definition understood by those skilled in the art. To the extern that the following description is of a specific embodiment or a particular use of the invention, it is intended to be illustrative only, and not limiting of the claimed invention. The following description is intended to cover all alternatives, modifications and equivalents that are included in the spirit and scope of the invention, as defined in the appended claims.

Embodiments include a peptide comprising an amino acid sequence having at least 65% identity with SEQ ID No. 1 (FETLRGDLRILSILRHQNLLKELQD) or a functional fragment thereof said peptide or functional fragment thereof being in a linear form or in an intramolecular macrocyclic form and composition comprising said peptide or a functional fragment thereof. The peptide may include at least a 25 amino acid sequence with at least 65%, 70%, 75%, 80%, 82%, 85%, 90%, 92%, 95%, 98%, 99% or 100% identity to SEQ ID NO:1 along the length of the 25 amino acid sequence. Determining percent identity of two amino acid sequences may include aligning and comparing the amino acid residues at corresponding positions in the two sequences. If all positions in two sequences are occupied by identical amino acid residues then the sequences are said to be 100% identical. Percent identity may be measured by the Smith Waterman algorithm (Smith T F, Waterman M S 1981 “Identification of Common Molecular Subsequences,” J Mol Biol 147: 195-197, which is incorporated herein by reference as if fully set forth). The peptide may have fewer than 25 residues of SEQ ID NO: 1.

A shorter peptide may have at least the sequence FETLRGDLRILSIL (SEQ ID No. 2). The peptide may include more than 25 amino acids. The peptide may have 8 or less, 7 or less, 6 or less, 5 or less, 4 or less, 3 or less, 2 or less, 1 or less, or zero amino acid replacement in comparison to the sequence of SEQID NO.1.

The replacement may be with any amino acid whether naturally occurring or synthetic. The replacement may be with an amino acid analog or amino acid mimetic that functions similarly to the naturally occurring amino acids. Naturally occurring amino acids are those encoded by the genetic code, as well as those amino acids that are later modified. The later modification may be but is not limited to hydroxyproline, y-carboxyglutamate, and O-phosphoserine modifications. Naturally occurring amino acids include the standard 20, and unusual amino acids. Unusual amino acids include selenocysteine. The replacement may be with an amino acid analog, which refers to compounds that have the same basic chemical structure as a naturally occurring amino acid; e.g., a carbon that is bound to a hydrogen, a carboxyl group, an amino group, and an R group. Examples of amino acid analogs include but are not limited to homoserine, norleucine, methionine sulfoxide, methionine methyl sulfonium. Such analogs may have modified R groups or modified peptide backbones. The amino acid analogs may retain the same basic chemical structure as a naturally occurring amino acid. The replacement may be with an amino acid mimetics, which refers to chemical compounds that have a structure that is different from the general chemical structure of an amino acid, but that functions similarly to a naturally occurring amino acid. The replacement may be with an a, a-disubstituted 5-carbon olefinic unnatural amino acid.

A replacement may be a conservative replacement, or a non-conservative replacement. A conservative replacement refers to a substitution of an amino acid with a chemically similar amino acid.

Conservative substitution tables providing functionally similar amino acids are well known in the art.

Such conservatively replacements include but are not limited to substitutions for one another: (1) Alanine (A), Glycine (G); (2) Aspartic acid (D), Glutamic acid (E); (3) Asparagine (N), Glutamine (Q); (4) Arginine (R), Lysine (K); (5) Isoleucine (1), Leucine (L), Methionine (M), Valine (V); (6) Phenylalanine (F), Tyrosine (Y), Tryptophan (W); (7) Serine (S), Threonine (T); and (8) Cysteine (C), Methionine (M) (see, e.g., Creighton, Proteins (1984)). A replacement may be from one amino acid to another with a similar hydrophobicity, hydrophilicity, solubility, polarity, or acidity.

A sequence having less than 100% identity to the reference sequence SEQ ID NO:1 may be referred to as a variant. An embodiment includes a composition including the peptide having a sequence that is a variant of SEQ ID NO: 1. An embodiment includes a composition including a stapled peptide having a sequence that is a variant of SEQ ID NO: 1 and having at least 10% activity of a stapled peptide (5). The activity may be determined by the binding to integrin αvβ6 and αvβ8 or by peptide stability assay in below Examples.

In an embodiment, one or more amino acids residues are replaced with a residue having a crosslinking moiety. The peptide may include at least a 25 amino acid sequence with the sequence SEQ ID NO:1, where two, one, or zero amino acid residues are replaced by a residue(s) having a cross linking moiety or are modified to include a cross-linking moiety. The peptide may include a crosslink from an amino acid side chain to another amino acid side chain within the 25 amino acid sequence. The peptide may include a crosslink from an amino acid side chain to the peptide backbone within the 25 amino acid sequence.

As used herein, a “peptide” or “polypeptide” comprises a polymer of amino acid residues linked together by peptide (amide) bonds. The term(s), as used herein, refer to proteins, polypeptides, and peptide of any size, structure, or function. Typically, a peptide or polypeptide will be at least three amino acids long. A peptide or polypeptide may refer to an individual protein or a collection of proteins. The peptides of the instant invention may contain natural amino acids and/or non-natural amino acids (i.e., compounds that do not occur in nature but that can be incorporated into a polypeptide chain). Amino acid analogs as are known in the art may alternatively be employed. One or more of the amino acids in a peptide or polypeptide may be modified, for example, by the addition of a chemical entity such as a carbohydrate group, a hydroxy! group, a phosphate group, a farnesyl group, an isofamesyl group, a fatty acid group, a linker for conjugation, functionalization, or other modification. A peptide or polypeptide may also be a single molecule or may be a multi-molecular complex, such as a protein. A peptide or polypeptide may be just a fragment of a naturally occurring protein or peptide. A peptide or polypeptide may be naturally occurring, recombinant, or synthetic, or any combination thereof.

A large number of agents are developed to target cellular contents, cellular compartments, or specific protein, lipid, nucleic acid or other targets or biomarkers within cells. While these agents can bind to their intracellular targets with strong affinity, many of these compounds, whether they be molecules, proteins, nucleic acids, peptides, nanoparticles, or other intended therapeutic agents or diagnostic markers cannot cross the cell membrane efficiently or at all.

This disclosure also provides cell-permeable and/or stable stapled peptides that can serve as efficient carriers of a broad range of cargoes (e.g., diagnostic agents or therapeutic agents) into living cells.

These universal carriers can provide cellular penetrance to cell-impermeable compounds or materials, and transport diverse cargoes to intracellular targets for therapeutic and diagnostic purposes. In some embodiments, the carrier is any cell-permeable stapled peptide. In other embodiments, the carrier is an internally cross-linked peptide that contains at least four guanidinium groups or at least four amino groups, wherein the peptide is cross- linked by a hydrocarbon staple or any other staple (e.g., a lactam staple, a UV- cycloaddition staple, a disulfide staple, an oxime staple, a thioether staple, a photoswitchable staple, a triazole staple, a double-click staple, a bis-lactam staple, or a bis-arylation staple).

The present disclosure provides cell-permeable and/or stable stapled peptides. These peptides can be used as carriers to transport various agents to or within a cell, e.g., to intracellular targets. These cell-permeable peptides are structurally stabilized. Structurally stabilized peptides comprise at least two modified amino acids joined by an internal (intramolecular) cross-link (or staple). Stabilized peptides as described herein include stapled peptides, stitched peptides, peptides containing multiple stitches, peptides containing multiple staples, or peptides containing a mix of staples and stitches, as well as peptides structurally reinforced by other chemical strategies (see. e.g., Balaram P. Cur. Opin. Struct.

Biol. 1992; 2:845; Kemp DS, et al, J. Am. Chem. Soc. 1996; 118:4240; Omer BP, et al, J. Am. Chem. Soc. 2001; 123:5382; Chin JW, et al, Int. Ed. 2001; 40:3806; Chapman RN, et al, J. Am. Chem. Soc. 2004;126: 12252; Home WS, et al, Chem., Int. Ed. 2008; 47:2853; Madden et al, Chem Commun (Camb). 2009 Oct 7; (37): 5588-5590; Lau et al, Chem. Soc. Rev., 2015,44:91-102; and Gunnoo et al, Org. Biomol. Chem., 2016,14:8002-8013; all of which are incorporated by reference herein in their entirety). In some instances, the peptides disclosed herein are stabilized by peptide stapling (see, e.g., Walensky, J. Med. Chem., 57:6275-6288 (2014), the contents of which are incorporated by reference herein in its entirety).

As used herein, “peptide stapling” is a term coined from a synthetic methodology wherein two side-chains (e.g., cross-linkable side chains) present in a polypeptide chain are covalently joined (e.g.,“stapled together”) using a ring-closing metathesis (RCM) reaction to form a cross-linked ring (see, e.g., Blackwell et al, J. Org. Chem., 66: 5291-5302, 2001; Angew et al, Chem. Int. Ed. 37:3281, 1994).

The term “peptide stapling” includes, e.g., the joining of two (e.g., at least one pair of) double bond-containing side-chains, triple bond-containing side- chains, or double bond-containing and triple bond-containing side chain, which may be present in a polypeptide chain, using any number of reaction conditions and/or catalysts to facilitate such a reaction, to provide a singly “stapled” polypeptide. The term “multiply stapled” polypeptides refers to those polypeptides containing more than one individual staple, and may contain two, three, or more independent staples of various spacing. Additionally, the term “peptide stitching,” as used herein, refers to multiple and tandem “stapling” events in a single polypeptide chain to provide a “stitched” (e.g., tandem or multiply stapled) polypeptide, in which two staples, for example, are linked to a common residue. Peptide stitching is disclosed, e.g., in WO 2008/121767 and WO 2010/068684, which are both hereby incorporated by reference in their entirety. In some instances, staples, as used herein, can retain the unsaturated bond or can be reduced. Stapling allows a polypeptide to maintain a constrained or discrete three-dimensional structure or ensemble of structures shape. The crosslinked peptide can increase hydrophobicity, cell permeability, and protease resistance. In some embodiments, the crosslinked peptide has a helical conformation (e.g., alpha helix).

In some embodiments, the cell-permeable stapled peptides can be any stabilized peptides that are permeable to cell membrane (e.g., enter the cell). In some embodiments, the cell-permeable stapled peptides have at least one staple and at least four guanidinium groups or amino groups. In some embodiments, the cell-permeable stapled peptide comprises a tracer (e.g., a fluorescent molecule such as TAMRA, FITC, etc.). Such molecules can be used for assessing cellular uptake of the stapled peptide (and its cargo).

In some embodiments, the cell-permeable stapled peptides of this disclosure have a consensus motif.

The sequence for the consensus motif is FETLRGDLRILSIL (SEQ ID No. 2). The staple positions can be joined by an internal hydrocarbon staple. In some embodiments, the staple positions can be joined by a nonhydrocarbon staple (e.g., ether, thioether, ester, amine, or amide, or triazole moiety). In some embodiments, the non-natural amino acids are 2-(4′-pentenyl) alanine, e.g., (S)-2-(4′-pentenyl) alanine.

In certain instances, the cell-permeable stapled peptide comprises a lactam staple, a UV-cycloaddition staple, a disulfide staple, an oxime staple, a thioether staple, a photo-switchable staple, a double-click staple, a bis-lactam staple, or a bis-arylation staple.

“Stapling” or “peptide stapling” is a strategy for constraining peptides typically in an alpha- helical conformation. Stapling is carried out by covalently linking the side-chains of two amino acids on a peptide, thereby forming a peptide macrocycle. Stapling generally involves introducing into a peptide at least two moieties capable of undergoing reaction to generate at least one cross- linker between the at least two moieties. The moieties may be two amino acids with appropriate side chains that are introduced into peptide sequence or the moieties may refer to chemical modifications of side chains.

Stapling provides a constraint on a secondary structure, such as an alpha- helical structure. The length and geometry of the cross-linker can be optimized to improve the yield of the desired secondary structure content. The constraint provided can, for example, prevent the secondary structure from unfolding and/or can reinforce the shape of the secondary structure. A secondary structure that is prevented from unfolding is, for example, more stable.

A “stapled peptide” is a peptide comprising a staple (as described in detail herein). More specifically, a stapled peptide is a peptide in which one or more amino acids on the peptide are cross-linked to hold the peptide in a particular secondary structure, such as an alphα-helical conformation. The peptide of a stapled peptide comprises a selected number of natural or non- natural amino acids, and further comprises at least two moieties which undergo a reaction to generate at least one cross-linker between the at least two moieties, which modulates, for example, peptide stability.

A “stitched” peptide, is a stapled peptide comprising more than one (e.g., two, three, four, five, six, etc.) staple.

In the present invention the peptide of SEQ ID No. 1 or functional fragment thereof may be stapled according to any known method in the art, for instance as described herein and in Methods Enzymol. 2012; 503:3-33. doi: 10.1016/B978-O-12-396962-0.00001-X. Stapled peptides for intracellular drug targets (Verdine GL, Hilinski GJ) incorporated by reference.

The composition may include a pharmaceutically acceptable carrier. The pharmaceutically acceptable carrier may include but is not limited to at least one of ion exchangers, alumina, aluminium stearate, lecithin, serum proteins, human serum albumin, buffer substances, phosphates, glycine, sorbic acid, potassium sorbate, partial glyceride mixtures of saturated vegetable fatty acids, water, salts, electrolytes, protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, waxes, polyethylene glycol, starch, lactose, dicalcium phosphate, microcrystalline cellulose, sucrose, dextrose, talc, magnesium carbonate, kaolin; non-ionic surfactants, edible oils, physiological saline, bacteriostatic water, Cremophor ELT^{M}(BASF, Parsippany, N.J.), and phosphate buffered saline (PBS).

The composition may include at least two different peptides in combination. For example, the composition may include the peptide (4) and the peptide (5).

Administering may include delivering a dose of 10 to 100 mg/kg/day of the peptide. The dose may be any value between 10 and 100 mg/kg/day. The dose may be any dose between and including any two integer values between 10 to 100 mg/kg/day. The dose may be 10, 15, 20, 25, 30, 35, 40, 45, 50, 55, 60, 65, 70, 75, 80, 85, 90, 95, or 100 mg/kg/day or any dose in a range between any two of the foregoing. Administering may include delivering any dose of a complementing therapeutic. The complementing therapeutic dose may be any 25 to 100 mg/kg/day. The complementing therapeutic dose may be any value between 25 and 100 mg/kg/day. The complementing therapeutic dose may be any dose between and including any two integer values between 25 and 100 mg/kg/day. The complementing therapeutic dose may be 10, 15, 20, 25, 30, 35, 40, 45, 50, 55, 60, 65, 70, 75, 80, 85, 90, 95, or 100 mg/kg/day or any dose in a range between any two of the foregoing. The complementing therapeutic may be any one or more of nanoparticle (e.g. gold nanoparticles, liposomes), a therapeutic agent (e.g. cytokines, chemotherapeutic drugs, antibodies and antibody fragments, toxins, nucleic acids), a diagnostic agent (e.g. radioactive compounds, fluorescence compounds, chemiluminescent compounds), a contrasting agent (e.g. microbubbles), or cellular components (e.g. chimeric antigen receptors or TCRs). The concentration of the peptide(s) and at least one complementing therapeutic in the composition may be set to deliver the daily dosage in a single administration, two-point administrations, multiple point administrations, or continuous administration (e.g., intravenously or transdermally) over a period of time. The period may be one day. The period may be 1, 2, 4, 8, 12, or 24 hours or a time within a range between any two of these values.

A composition including peptide of the invention may include any amount of the peptide. The amount may be that sufficient to deliver the dosage as set forth above in a suitable volume or sized delivery mode. When the dosage is split into multiple administrations throughout a time period, the amount in one volume or delivery mode may be the total dosage divided by the number of administrations throughout the time period. When present in a composition, the complementing therapeutic may be at any complementing therapeutic amount. Like the peptide, the complementing therapeutic amount may be tailored to deliver the right complementing therapeutic amount in the volume or delivery mode used for administration.

The patient may be an animal. The patient may be a mammal. The patient may be a human. The patient may be a cancer patient. The cancer patient may be a oral or skin squamous cell carcinoma, head and neck, pancreatic, ovarian, lung, cervix, colorectal, breast cancer, brain tumors (e.g. glioblastoma and astrocytoma) cancer patient.

The route for administering a composition or pharmaceutical composition may be by any route. The route of administration may be any one or more route including but not limited to oral, injection, topical, enteral, rectal, gastrointestinal, sublingual, sublabial, buccal, epidural, intracerebral, intracerebroventricular, intracisternal, epicutaneous, intraderm al, subcutaneous, nasal, intravenous, intraarterial, intramuscular, intracardiac, intraosseous, intrathecal, intraperitoneal, intravesical, intravitreal, intracavernous, intravaginal, intrauterine, extra-amniotic, transderm al, intratumoral, and transmucosal.

Embodiments include a method of making the peptides of the invention, including the stapled peptide. The method may include constructing a library including at least one modified peptide. The modified peptide may include at least a 25 amino acid sequence with at least with at least 65%, 70%, 75%, 80%, 82%, 85%, 90%, 92%, 95%, 98%, 99% or 100% identity to SEQ ID NO:1. The modified peptide may have an amino acid replacement at 8 or less, 7 or less, 6 or less, 5 or less, 4 or less, 3 or less, 2 or less, 1 or less, or zero positions in comparison to the sequence of SEQ ID NO: 1. The replacement(s) may be as described above.

The method may include screening the library for affinity of the at least one modified peptide toward integrin αvβ6 and/or αvβ8. Screening the library for affinity may include exposing the library to the integrin αvβ6 and/or αvβ8 under conditions effective for binding between the peptide and αvβ6 and/or αvβ8. A non-limiting example of conditions may be found in Example below.

The method may include selecting a modified peptide with affinity toward integrins αvβ6 and/or αvβ8 to obtain a selected modified peptide. Selecting may include isolating the highest affinity members from the library based on known methods in the art.

The method may include synthesizing a peptide having the sequence of the selected modified peptide. The peptide may include a crosslink from an amino acid side chain to another amino acid side chain within the 25 amino acid sequence. The peptide may include a crosslink from an amino acid side chain to the peptide backbone within the 25 amino acid sequence. The amino acid in the crosslink may be the same as in the selected modified peptide or altered to include a cross-link moiety.

The method may include evaluating the stability of the peptide. Methods and conditions for evaluating the stability of the peptide may be set forth in the Example below.

An embodiment includes a peptide or a peptide composition comprising a peptide consisting of, consisting essentially of, or comprising the sequence of any amino acid sequence herein. The peptide composition may include any complementing therapeutic herein. The peptide composition may include a pharmaceutically acceptable carrier. The peptide or peptide composition may be used in a method of treating or diagnosing a disease, in particular fibrosis or cancer by administering the peptide or peptide composition to patient in need thereof. The dosage of peptide in the peptide composition for the method may be like that of the peptide in the method described above. The dosage of complementing therapeutic in the method may be like that of the complementing therapeutic in the method described above.

### Examples

The inventors investigated the structural determinants of 1/αvβ6 interaction by heteronuclear 2D-NMR STD methods and docking calculations. Intriguingly, while 1 is highly specific for αvβ6, reconstitution of the canonical RGDLXXL motif, combined with a click-chemistry stapling strategy results in a novel potent ligand suitable for the dual targeting of αvβ6/αvβ8 for diagnostic and therapeutic purposes.

The inventors studied the conformation of recombinant peptide 1 in physiological conditions by homonuclear and heteronuclear multidimensional NMR. Peptide 1 was expressed in E. Coli as insoluble fusion partner of ketosteroid isomerase, subsequently cleaved with CNBr and purified by HPLC.22 Recombinant 13C/15N 1 displays the typical NOE pattern of α-helical conformation between residue E46 to K59, with both termini being unstructured (FIG. 1a, c, Tables 1-2).

TABLE 1
NMR experiments. List of 2D and 3D NMR experiments performed for the
characterization of the peptides, where TD is the total number of
points acquired, SI the total number of points used for processing
after zero filling, SW the spectral width, and tₘᵢₓ the mixing time.

| | | | | SW | tₘᵢₓ | solvent |
|---|---|---|---|---|---|---|
| Experiment | Pulse sequence | TD | SI | (ppm) | (ms) | suppression |
| ¹H-¹H-TOCSY^{a} | Mlevesgpph | 2048 x 400 | 2048 x 1024 | 11 x 11 | 60 | excitation |
| | (SEQ ID No. 3) | | | | | sculpting |
| ¹H-¹H-NOESY | Noesyesgpph | 2048 x 512 | 2048 x 1024 | 11 x 11 | 200-400 | excitation |
| | (SEQ ID No. 4) | | | | | sculpting |
| ¹H-¹H-NOESY | Noesyesgpph | 2048 x 512 | 2048 x 1024 | 11 X 11 | 200 | excitation |
| (100% D₂O) | (SEQ ID No. 5) | | | | | sculpting |
| ¹H-¹³C-HSQC | hsqcetgpsisp2 | 2048 x 256 | 2048 x 512 | 11 x 80 | — | gradient |
| | (SEQ ID No. 6) | | | | | selection |
| ¹H-¹⁵N-HSQC | hsqcfpf3gpphwg | 2048 x 256 | 2048 x 512 | 11 x 21 | — | water gate |
| | (SEQ ID No. 7) | | | | | |
| 3D-HNCA | hncagp3d | 2048 x 60 x 110 | 2048 x 256 x 256 | 11 x 21 x 32 | — | gradient |
| | (SEQ ID No. 8) | | | | | selection |
| 3D-HNCO | hncogp3d | 2048 x 60 x 72 | 2048 x l28 x 128 | 11 x 21 x 22 | — | gradient |
| | (SEQ ID No. 9) | | | | | selection |
| 3D-HNHA | hnhagp3d | 2048 x 180 x 46 | 2048 x 256 x 128 | 12 x 12 x 21 | — | gradient |
| | (SEQ ID No. 10) | | | | | selection |
| ¹H-¹³C-HSQC- | noesyhsqcetgp3d | 2048 x 46 x 176 | 2048 x l28 x 512 | 11 x 27 x 11 | 180 | gradient |
| NOESY | (SEQ ID No. 11) | | | | | selection |
| (100% D₂O) | | | | | | |
| ¹H-¹⁵N-HSQC- | noesyhsqcetf3gp3d | 2048 x 44 x 200 | 2048 x 256 x 512 | 12 x 21 x 12 | 120 | gradient |
| NOESY^{b} | (SEQ ID No. 12) | | | | | selection |
| ¹H-¹⁵N-HSQC | hsqct1etf3gpsi3d | 2048 x 110 | 2048 x 256 | 11 x 21 | — | gradient |
| (T₁) | (SEQ ID No. 13) | | | | | selection |
| ¹H-¹⁵N-HSQC | hsqct2etf3gpsi3d | 2048 x 110 | 2048 x 256 | 11 x 21 | — | gradient |
| (T₂) | (SEQ ID No. 14) | | | | | selection |
| ¹H-¹⁵N-HSQC | hsqcnoef3gpsi | 2048 x 200 | 2048 x 512 | 11 x 21 | — | gradient |
| (hetNOE) | (SEQ ID No. 15) | | | | | selection |

TABLE 2
Statistics of the 30 lowest energy structures of 1. Ramachandran quality
parameters were assessed using the PROCHECK-NMR software.[24]
Restraints information

| | |
|---|---|
| Total number of experimental distance | 360 |
| restraints | |
| NOEs (intraresidual/sequential/short/medium) | 112/121/115/12 |
| Dihedral angle restraints (phi/psi) | 7/7 |

Deviation from idealized covalent geometry

| | |
|---|---|
| Bonds (Å) | 0.0026 ± 0.0001 |
| Angles (°) | 0.434 ± 0.016 |

Coordinate r.m.s.d. (A)^{a}

| | |
|---|---|
| Ordered backbone atoms (N, Cα, CO) | 0.208 |
| Ordered heavy atoms | 0.715 |

Ramachandran quality parameters

| | |
|---|---|
| Residues in most favored regions (%) | 79.2 (100) ^{b} |
| Residues in additional allowed regions (%) | 19.1 |
| Residues in generously allowed regions (%) | 0.9 |
| Residues in generously allowed regions (%) | 0.9 |

^{a}Root mean square deviation between the ensemble of structures and the lowest energy structure calculated on residues E46 to N56.
^{b} Values obtained for residues E46-N56.

Accordingly, the helical segment and both termini display relatively high (˜ 0.5) and very low (<0.3) heteronuclear NOE values, respectively (FIG. 1d). The RGD motif adjacent to the a-helix is relatively flexible, thus well suited to adapt inside the integrin-binding pocket (FIG. 1a). The first three turns of the post-RGD helix are amphipathic, with 148, L49, 151, L52 and E46, R47, S50 on opposite sides (FIG. 1a, b). Peptide 1 propensity to adopt an α-helical conformation is in line with previous NMR studies on CgA47-66, an antifungal CgA-derived peptide, all-helical in the helix-promoting solvent trifluoro-ethanol, TFE.23 To gain molecular insights into 1/αvβ6 complex and group selective information on the interaction, the inventors performed in the presence of the extracellular region of recombinant human αvβ6 (4 μM) 1D-¹H Saturation Transfer Difference (STD) spectroscopy (FIG. 5a) and heteronuclear two-dimensional STD experiments,24 exploiting isotopically labelled (13C/15N) recombinant peptide 1 (0.5 mM) (FIG. 2a, FIG. 5b). The 2D-STD-1H-1⁵N-HSQC resolved peak ambiguities in the 1D-¹H STD spectrum and provided residue-specific STD effects values. Hydrophobic amino acids (148, L49, 151, and L52) of the post-RGD helix displayed the strongest STD % values[25] (>75%), suggesting their important contribution to receptor binding (FIG. 2b). Intense STD effects of the methyl groups of branched amino acids in 2D-STD-1H-¹³C-HSQC corroborated their involvement in the interaction, though signal overlap hampered their quantification for epitope mapping (FIG. 5b).

To exclude false positive effects, the inventors spiked recombinant peptide 1 with bovine serum albumin as negative control: no interaction occurred, and the inventors did not observe any STD signal (FIG. 6a, b). 2D-STD-1H-15N-HSQC performed with αvβ6 pre-incubated with 20 mM of EDTA resulted in depletion of the STD effects, thus confirming the presence of the electrostatic clamp between the receptor metal ion and the aspartate side chain of the RGD motif (FIG. 6c). This result is in line with competitive binding assays using a peptide with RGE instead of RGD (2), yielding a Ki >50 μM (Tables 3a and 3b).

TABLE 3a
Inhibition constants (Ki, nM) and the associated standard error of the mean of
compounds 1-6 for integrins as determined by competitive binding assay.

| | | αvβ6 | | αvβ8 | | α5β1 | | αvβ5 | | αvβ3 | |
|---|---|---|---|---|---|---|---|---|---|---|---|
| Code | Peptide^{a} | n^{b} | Ki | n | Ki | n | Ki | n | Ki | n | Ki |
| l | FETLRGDERILSILRHQNLLKELQD | 6 | 15.5 ± 3.2 | 6 | 7663 ± 1704 | 4 | 9206 ± 1810 | 5 | 3600 ± 525 | 4 | 2192 ± 1690 |
| | (SEQ ID No. 16) | | | | | | | | | | |
| 2 | FETLRG [symbol] ERILSILRHQNLLKELQD^{c} | 1 | >50000 | 1 | >50000 | 1 | >50000 | 1 | >50000 | 1 | >50000 |
| | (SEQ ID No. 17) | | | | | | | | | | |
| 3 | FETLRGDERILSILR | 4 | 277 ± 74^{d} | 1 | 31174 | 1 | 10110 | 1 | 2039 | 1 | 1250 |
| | (SEQ ID No. 18) | | | | | | | | | | |
| 4 | FETLRGD [symbol] RILSILRHQNLLKELQD | 11 | 1.6 ± 0.3^{e} | 6 | 8.5 ± 3.7^{f} | 3 | 924 ± 198 | 4 | 2405 ± 1592 | 3 | 1928 ± 226 |
| | (SEQ ID No. 1) | | | | | | | | | | |
| 5 | FETLRGD [symbol] RILSILR [symbol] QNL [symbol] KELQD | 7 | 0.6 ± 0.1^{g} | 6 | 3.2 ± 1.2^{h} | 3 | 1310 ± 389 | 4 | 2741 ± 615 | 3 | 2453 ± 426 |
| | (SEQ ID No. 19) | | | | | | | | | | |
| 6 | NAVPNLRGDLQVLAQKVART | 8 | 0.9 ± 0.2 | 6 | 69 ± 12 | 5 | 2317 ± 10 | 5 | 15449 ± 2418 | 3 | 26197 ± 7387 |
| | (SEQ ID No. 20) | | | | | | | | | | |

^{a}Mutated residues and triazole-stapled residues (X1 and X2, as defined in FIG. 9a).
^{b}n, number of independent experiments (each performed with 6 different concentrations of competitor in technical duplicates),
^{c}Ki of 2 as determined in [21].
^{d}P value versus 1: p < 0.05; two tailed t test,
^{e}P value versus 1: p < 0.001, two tailed t test,
^{f}P value versus 1: p < 0.01, two tailed t test,
^{g}P value versus 4: p < 0.05; two tailed t test.
^{h}P value versus 4: p > 0.1; two tailed t test.

TABLE 3b
Binding affinity of peptide 2a, 5, 5a and 6 for
αvβ6 and αvβ8 integrins (Ki values, mean ± SE),
as determined by the competitive binding assay.

| | | Binding affinity for | | | |
|---|---|---|---|---|---|
| | | αvβ6 | | αvβ8 | |
| | Peptides or | | Ki | | Ki |
| Code | compounds^{a} | n^{a} | (nM) | n^{a} | (nM) |
| | CgA-derived | | | | |
| | peptides | | | | |
| 2a | CFETLRGEERILSIL | 2 | >50000 | 2 | >50000 |
| | RHQNLLKELQD | | | | |
| | (SEQ ID No. 36) | | | | |
| 5 | FETLRGDLRILSILR | 7 | 0.6 ± 0.1 | 6 | 3.2 ± 1.2 |
| | X₁QNLX₂KELQD | | | | |
| | (SEQ ID No. 19) | | | | |
| 5a | CFETLRGDLRILSIL | 4 | 1.5 ± 0.06 | — | — |
| | RX₁QNLX₂KELQD | | | | |
| | (SEQ ID No. 23) | | | | |
| | Foot and mouth | | | | |
| | disease virus- | | | | |
| | derived peptide | | | | |
| 6 | NAVPNLRGDLQVLAQ | 8 | 0.9 ± 0.2 | 6 | 69 ± 0.2 |
| | KVART | | | | |
| | (SEQ ID No. 20) | | | | |

^{a}n, number of independent experiments (each performed with 6 different concentrations of competitor in technical duplicates)

The precursor of 5 is a peptide in which positions 54 and 58 are occupied by L-propargylglycine (Prg) and L-e-azido-norleucine [Nle(εN3)] (LysN3), respectively. Click chemistry generates a single triazole-stapled peptide, i.e. 5 (see FIG. 9a) Next, the inventors incorporated the 2D-STD experimental information in data driven docking calculations (HADDOCK2.2)25 to determine the binding mode of 1 with the extracellular head of αvβ6 (PDB: 5FFO).[16] The model highlights receptor ligand-interactions highly reminiscent of those observed for proTGF-P1/αvβ6 complex (FIG. 2c, FIG. 7a).16 On one hand the guanidinium of R43 forms electrostatic interactions with Asp218av and Asp150av, on the other hand the carboxylate of D45 coordinates the metal ion-dependent adhesion site (MIDAS) and interacts with the amide of Ser1270_{β6} and Asn2180_{β6}. 148, L49, 151, L52 located respectively on the second and the third turn of the post-RGD amphipathic a-helix, make extensive hydrophobic interactions with P6 residues of the specificity determining loops (SDLs), including Ala1260₆, Asp1290_{β6} (SDL1), lle1830₆, Tyr1850_{β6} (SDL2), Ala2170_{β6} (SDL3) (FIG. 2c), thus explaining the selectivity of 1 towards αvβ6 with respect to the other av integrins (Table 3). Since in inventors' model residue E46 points towards the receptor interior, the inventors reasoned that the preformed a-helix of 1 might entropically compensate the unfavourable electrostatic contribution of the negative charge within the hydrophobic binding pocket. Thus, the inventors synthetized a shorter peptide containing the hydrophobic residues important for the interaction, without ten C-terminal residues supposed to be crucial for the helical propensity (3).

Indeed, 3 showed a drastic reduction both in α-helical content (FIG. 8) and binding to αvβ6 (Ki: 277±74 nM) (Table 3), supporting the notion that the stability of the preformed four-turn amphipathic helix adjacent to the RGD motif is fundamental for effective αvβ6 recognition.[18,26] The inventors next predicted that restoring of the canonical LXXL motif might increase the affinity of 1 for αvβ6. Indeed, the replacement in position D+1 of E46 with a leucine (4) lowered the Ki by one order of magnitude (Ki: 1.6±0.3 nM) (Table 3). Intriguingly, reconstitution of the LXXL motif transforms 4 into a bi-selective ligand able to bind also αvβ8 (Ki: 8.5±3.7 nM).

Structurally, αvβ6 and αvβ8 share a similar wide lipophilic SDL pocket, suitable for hydrophobic interactions with the amphiphilic helix of 4. Of note, minor changes in the shape and in the sequence of the SDL loops, such as the presence in SDL2 of K170 and T171 in 36 and S159 and 1160 in 38, respectively (FIG. 7c) might explain why the presence of E46 in the ligand is tolerated by 36 and not by 38 (Table 3a). Prompted by these results, the inventors hypothesized that chemical stabilization of the a-helix via stapling, i.e. “side-chain-to-side-chain” cyclization[27], might further improve the binding properties of 4. Based on a 5/αvβ6 model (FIG. 2d) the inventors constrained this peptide via a triazole-bridged macrocyclic scaffold between residues in position 54 (propargylglycine) and 58 (azidolysine) through copper-catalyzed azide-alkyne cycloaddition (5) (FIG. 9a, b) [27,28]. Indeed, the structural constrain boosted the α-helical content of 5, compared to 4, (FIG. 9c), resulting in a significant 2 to 3-fold increase in αvβ6 binding (Ki: 0.6±0.1 nM), comparable to the reference compound foot and mouth disease virus-derived peptide A20FMDV2 (6, Ki: 0.9±0.2 nM) (Table 3) [11,18]. Stapling maintained nM binding to αvβ8 (Ki: 3.2±1.2 nM), thus generating, to the best of inventors' knowledge, the strongest bi-selective ligand for αvβ6/αvβ8 described so far[6,29].

Importantly, peptides 1, 4, 5 and 6 were able to recognize αvβ6 in its physiological context, as they bound cell-surface expressed αvβ6 on human bladder cancer 5637 cells and human keratinocytes (HaCat) with a relative binding potency similar to the one observed with purified recombinant αvβ6 (FIG. 10). 5 was the most effective with an activity comparable to the reference 6 (FIG. 3a, FIG. 11) [30]. Notably, both 4 and 5 were not cytotoxic in vitro (FIG. 12). To assess whether 5 was suitable for nanoparticle functionalization and delivery to cancer cells, the inventors coupled it to fluorescent quantum dot nanoparticles via an N-terminal cysteine (5-Qdot) and evaluated its binding to 5637 cells.

Flow cytometry and fluorescence microscopy showed that 5-Qdot, but not a control nanoconjugate without the targeting ligand (*Qdot), bound the cells, indicating that 5 maintains its receptor-tailored properties also after conjugation (FIG. 3b, c). Finally, ELISA stability assays of 4 and 5 conjugated to horseradish peroxidase (4-HRP, 5-HRP) in human serum indicated that >50% of 4-HRP and 5-HRP were still present after 24 hours of incubation at 37° C., supporting their proteolytic stability in biological fluids (FIG. 13). Importantly, stability assays with mouse liver microsomes showed that 5 was more stable than 4 (t1/2=4.3 h and t1/2=1.3 h, respectively, FIG. 14).

In conclusion, NMR experiments allied to computational and biochemical methods elucidated the molecular details at the basis of αvβ6 recognition by CgA-derived peptides, giving first hints on the interaction between αvβ6 and CgA [19]. The entropic gain, deriving from the preformed four-turns a-helix adjacent to the RGD motif, combined to the hydrophobic interactions between residues in position D+3, D+4, and D+7 and the 36 subunit, largely compensate the unfavourable electrostatic repulsion of E46 in position D+1. Thus, the natural αvβ6 recognition motif RGDLXXL is less restrictive than previously supposed and can be extended to RGDEXXL, provided that the helix adjacent to RGD is preformed and presents an extensive hydrophobic surface for αvβ6 interaction. Importantly, the complex model inspired the design of novel peptides, including a stapled one with high stability, sub-nanomolar affinity and bi-selectivity for αvβ6/αvβ8 integrins. These molecules, derived from a human protein, may represent useful and safer tools for the ligand-directed targeted delivery of diagnostic and therapeutic compounds and nanodevices to epithelial cancers with high expression of αvβ6 and/or αvβ8, such as oral and skin squamous cell carcinoma.¹³Furthermore, in light of the roles of both αvβ6 and αvβ8 in TGFβmaturation and fibrosis,1 the dual targeting ability of these compounds could be also conveniently used to develop anti-fibrotic drugs and tracer devices, thus adding to the still limited number of small molecules able to specifically recognize these integrins.

### MATERIAL AND METHODS

### Reagents

Recombinant human αvβ6 was from R&D Systems (Minneapolis, Minn.); wild-type human integrins αvβ3, αvβ5, and a5@1 (octyl P-D-glucopyranoside preparation) were obtained from Immunological Sciences (Rome, Italy); anti-αvβ6 monoclonal antibody (clone 10D5, IgG2a) was from Millipore (Billerica, Mass.); anti-αvβ8 polyclonal antibody (EM13309, IgGs) was form Absolute Antibodies (Oxford, UK); normal rabbit immunoglobulins (IgGs, purchased from Primm, Italy) were purified by affinity chromatography on protein A-sepharose; mouse IgG1, (clone MOPC 31C) was from Sigma (Missouri, USA); goat anti-mouse and goat anti-rabbit Alexa Fluor 488-labeled secondary antibodies were purchased from Invitrogen.

Expression and purification of recombinant “C/¹⁵N peptide 1 Preparation of the expression vector coding for recombinant peptide 1 Recombinant peptide 1 was produced by recombinant DNA technology as a fusion product with ketosteroid isomerase (KSI), by cloning the peptide 1 sequence downstream the KSI gene and upstream of a His(6x)-tag sequence. 5′-phosphorylated forward and reverse complementary DNA oligonucleotides coding for peptide 1 were synthesized by PRIMM (Italy).

Forward:
(SEQ. ID No. 21)
5′-TTTGAGACACTCCGAGGAGATGAACGGATCCTTTCCATTCTGAGACA
TCAGAATTTACTGAAGGAGCTCCAAGACATG-3′;
Reverse:
(SEQ. ID No. 22)
5′-GTCTTGGAGCTCCTTCAGTAAATTCTGATGTCTCAGAATGGAAAGGA
TCCGTTCATCTCCTCGGAGTGTCTCAAACAT-3′.

The oligonucleotides produced had a three-base 3′ overhangs (underlined) coding for a methionine residue, necessary for cloning strategy and for CNBr cleavage. The oligonucleotides (10 μM each) in 40 mM Tris-HCl pH 8.0, 50 mM NaCl, 10 mM MgCl₂ were annealed as follows: 10 min at 99° C., 15 min at 30° C. and 20 min at 4° C. The annealed product (0.26 pmol) was then ligated with 0.026 pmol of a pET31b(+) plasmid (Novagen), previously digested with AlwNI enzyme. E. coli cells (DH5a) were then transformed with the ligation product and ampicillin-resistant colonies were selected and screened for the correct incorporation of the insert by restriction digestion using Xhol and Xbal enzymes. The identity of the selected clone (called KSI-P1 plasmid) was confirmed by DNA sequencing (Eurofins Genomics, Germany). The KSI-P1 plasmid was then used to transform BL21 DE3 E. Coli cells for protein expression.

### Expression of the KSI-peptide 1 fusion protein

BL21 DE3 cells containing KSI-P1 plasmid were grown in 50 mL LB medium containing ampicillin (100 μg/ml) overnight at 37° C. under shaking. Five mL of overnight culture were then inoculated in 0.5 L of M9 medium supplemented with ampicillin, ¹³C-D-glucose (2 g) and 1⁵NH₄Cl (1.5 g), as unique sources of carbon and nitrogen, and left to grow at 37° C. under shaking. When the culture reached an optical density at 600 nm of 0.8 Units, 1 mM isopropyl P-D-1-thiogalactopyranoside was added to induce protein expression. The cells were then incubated for additional 16 h at 28° C. under shaking.

### Purification of KSI-Peptide 1 from Inclusion Bodies

The cells were pelleted, resuspended in 10 mL of lysis buffer (50 mM Tris-HCl pH 8, containing 10 mM EDTA, 0.1% Triton X-100, 20 μg/mL DNAse, 20 μg/mL RNAse and 50 μg/ml lysozyme) and broken by sonication using an Ultrasonic Processer (Sonopulse, Bandelin) (3 cycles of 1.5 minute each, alternating 30 s of pulses and wait periods). The cell lysate was centrifuged (14000 x g, 15 min 4° C.), and the resulting pellet was washed twice with washing buffer (50 mM Tris HCl pH 8, containing 10 mM EDTA and 0.5% Triton X-100) followed by two additional washes with water. The pellet was then resuspended with 20 ml of refolding buffer (20 mM Tris-HCl pH 8, containing 150 mM NaCl, 10 mM imidazole pH 8, 1 mM 2-mercaptoethan-1-ol, 6 M guanidinium chloride) and loaded onto a chromatography column filled with 10 ml of Ni”-NTA resin (Qiagen) (flow rate 0.5 ml/min at 4° C.), previously equilibrated with refolding buffer. The column was washed with 50 ml of refolding buffer followed by 50 ml of refolding buffer-1 (20 mM Tris-HCl pH 8, containing 150 mM NaCl, 20 mM imidazole pH 8, 1 mM 2-mercaptoethan-1-ol, 6 M guanidinium chloride); the protein was then eluted from the resin after incubation with 5 mL of elution buffer (20 mM Tris-HCl pH 8, containing 150 mM NaCl, 300 mM imidazole pH 8, 1 mM 2-mercaptoethanol, 6 M guanidinium chloride) at 4° C. for 15 minutes; small volumes of elution buffer were then gradually added until the protein was completely eluted (checked on small aliquots using Bradford reagent). The product (30 ml) was then dialyzed using a 3.5 kDa membrane (CelluSep) against 2 L of water at 4° C. for 16 h. The dialysis product, consisting of insoluble protein, was then centrifuged (500 x g, 30 min, at 4° C.), washed twice with water and stored at −80° C.

CNBr cleavage of KSI-peptide 1fusion protein and purification of recombinant peptide 1 The pellet containing recombinant KSI-peptide 1 was dissolved with 5 ml of TFA (80%, v/v) containing 0.2 g of CNBr, and stirred for 18-24 h at 25° C. in the dark. CNBr cleavage led to the formation of a peptide with the expected N-terminal residue and with a homoserine lactone residue at the C-terminus (FIG. 15a). The solution was partially evaporated by bubbling N₂ gas, diluted with 5 volumes of water, and freeze-dried. The product was resuspended in 20 mM phosphate buffer. pH 7.5-8, 100 mM NaCl and left to stir at 25° C. for 16 h in the dark. The suspension was then centrifuged at 14000×g for 15 min. The resulting supernatant was loaded onto a preparative Shimadzu Shim-pack G15 column and purified as described below. Fractions with a purity >95% were pooled and lyophilized. Product identity were assessed by mass spectrometry (FIG. 15b,c).

### Synthetic Peptides

Peptides Synthesis

Peptides 3, 4 and the linear peptide precursor of 5 (with propargylglycine and azidolysine in positions 54 and 58, respectively, FIG. 9a) were assembled by stepwise microwave-assisted Fmoc-SPPS on a Biotage ALSTRA Initiator+peptide synthesizer, operating in a 0.12 mmol scale on a Rink-amide resin (0.5 mmol/g). Resin was swelled prior to use with an NMP/DCM mixture. Activation and coupling of Fmoc-protected amino acids were performed using Oxyma 0.5 M/DIC 0.5 M (1:1:1), with a 5 equivalent excess over the initial resin loading. Coupling steps were performed for 7 min at 75° C.

Deprotection steps were performed by treatment with a 20% piperidine solution in DMF at room temperature (1×10 min). Following each coupling or deprotection step, peptidyl-resin was washed with DMF (4×4 ml). Upon complete chain assembly, peptides were cleaved from the resin using a 90% TFA, 5% water, 2.5% thioanisole, 2.5% TIS (triisopropyl silan) mixture (2 hours, room temperature).

Following precipitation in cold diethyl ether, crude peptide was collected by centrifugation and washed with additional cold diethyl ether to remove scavengers. Peptides were then dissolved in 50% acetonitrile containing 0.07% TFA and purified by preparative RP-HPLC.

Synthetic peptides 1, 2 and 6 were purchased from Biomatik (Delaware, USA), peptide 6 (Tables 3 and 4) was purchased from Biomatik (Delaware, USA). Peptide identity and purity were confirmed by mass spectrometry analysis and reverse-phase HPLC. Peptide concentration was determined using the Ellman's assay.

Intramolecular Cu -catalyzed azido-alkyne 1,3- cycloaddition to obtain 5

To the linear peptide precursor of 5 (with propargylglycine and azidolysine in positions 54 and 58, respectively) (0.5 mg/ml in degassed water) were added CuS_{O} 5 H₂O (10 eq) and ascorbic acid (10 eq) in order to originate in situ Cu catalyst (FIG. 9a). The reaction was left to stir at room temperature until the complete conversion of linear precursor into the desired heterodetic 1,2,3-triazolyl-containing peptide occurred (monitoring by RP-HPLC, FIG. 9b, 15b). The resulting stapled peptide was RP-HPLC purified as described below.

Peptides purification and characterization

Peptides were purified by reversed phase high performance liquid chromatography (RP-HPLC) using a Shimadzu Prominence HPLC system, equipped with a Shimadzu Prominence preparative UV detector, connected to Shimadzu Shim-pack G15 10p C18 90A (250×20 mm). The column was eluted with mobile phase A (3% acetonitrile, 0.07% trifluoroacetic acid in water) and mobile phase B (70% acetonitrile, 0.07% trifluoroacetic acid in water) using the following chromatographic method: 0% B (7 min), linear gradient (0-30% B), 40 min; flow rate, 14 ml/min. Peptides purity was >95% as determined by analytical RP-HPLC using a Shimadzu Shim-pack GWS 5p C18 90A column (150×4.6 mm) connected to diode array detector (FIG. 15b, 16). Peptides identity was confirmed by mass spectrometry analysis (Table 4).

TABLE 4
Molecular mass of synthetic peptides as
determined by mass spectrometry analysis
(ESI-MS).

| | | Monoisotopic | |
|---|---|---|---|
| | | mass (Da) | |
| Peptides^{a} | Code | Expected | Found |
| CgA derived peptides | | | |
| FETLRGDERILSILRHQNLLKELQD | 1 | 3035.7 | 3035.5 |
| (SEQ ID No. 16) | | | |
| FETLRGEERILSILRHQNLLKELQD | 2 | 3049.7 | 3051.0 |
| (SEQ ID No. 17) | | | |
| CFETLRGEERILSILRHQNLLKELQD | 2a | 3022.5 | 3022.5 |
| (SEQ ID No. 36) | | | |
| FETLRGDERILSILR | 3 | 1817.0 | 1817.4 |
| (SEQ ID No. 18) | | | |
| FETLRGDLRILSILRHQNLLKELQD | 4 | 3022.5 | 3022.5 |
| (SEQ ID No. 1) | | | |
| FETLRGDLRILSILRX₁QNLX₂KELQD^{b} | 5 | 3059.7 | 3059.7 |
| (SEQ ID No. 19) | | | |
| CFETLRGDLRILSILRX₁QNLX₂KELQD^{d} | 5a | 31201.7 | 3120.7 |
| (SEQ ID No. 23) | | | |
| Foot and mouth disease | | | |
| virus-derived peptide | | | |
| NAVPNLRGDLQVLAQKVART^{c} | 6 | 2163.2 | 2162.4 |
| (SEQ ID No. 20) | | | |

^{a}Single letter code; mutated residues (italics, bold); triazole-stapled residues (bold X1 and X2, propargylglycine and azidolysine respectively).
^{b}N-terminal acetylated and C-terminal amidated.
^{c}Also known as A20FMDV2 peptide.
^{d}C-terminal amidated

### NMR Experiments

NMR spectra were recorded on a Bruker Avance-600 spectrometer (Bruker BioSpin) equipped with a triple-resonance TC cryo-probe with an x, y, z shielded pulsed-field gradient coil. All the spectra were acquired at 280 K. Peptides were dissolved in NMR buffer (20 mM phosphate buffer pH 6.5, 100 mM NaCl, 20 mM M9Cl₂, 0.5 mM CaCl₂, 90% H₂O, 10% D₂O or 100% D₂O ) to a concentration of 0.5-1 mM. Each sample was transferred in a 3 mm NMR tube for NMR analysis. Resonance assignments were obtained from the analysis of two-dimensional homonouclear (2D ¹H-¹H TOCSY, TOtal Correlation SpectroscopY, tₘᵢₓ =60 ms; 2D ¹H-¹H NOESY, Nuclear Overhauser Effect SpectroscopY, tₘᵢₓ =100-600 ms) and heteronuclear (2D-1H-¹³C-HSQC, Heteronuclear Single Quantum Coherence, 2D ¹H-¹³C HMBC, Heteronuclear Multiple Bond Correlation) experiments (Table 1).

Complete ¹H, ¹³C and 1⁵N resonance assignment of recombinant peptide 1 was obtained from the following 2D and three-dimensional (3D) experiments acquired for ¹³C/1⁵N recombinant peptide 1: 2D-¹H-1⁵N-HSQC, 3D-HNCA, 3D-HNCO. 3D-1H-¹³C-HSQC-NOESY (tₘᵢₓ =180 ms) and 3D-1H-1⁵N-HSQC-NOESY (tₘᵢₓ =120 ms) (Table 1). All spectra were processed using Topspin 3.2 NMR software from Bruker.

Spectral analysis was performed using CCPNmr Analysis2.4 software.[31] Chemical shifts of recombinant peptide 1 have been deposited in BioMagResBank (accession code 34381). Chemical shift assignment of 3, 4 and 5 are reported in Tables 5-7.

TABLE 5
Chemical shift assignment of 3. Assignment was determined in 20
mM phosphate buffer, 100 NaCl mM, pH 6.5 (10% D₂O) at 280K.

| Residue | HN | Hα | Hβ | Hγ | | Hδ | Hε/HNε |
|---|---|---|---|---|---|---|---|
| F39 | — | 4.28 | 3.25 | — | | 7.26 | — |
| | | | 3.15 | | | | |
| E40 | 8.76 | 4.45 | 2.03 | 2.24 | | — | — |
| | | | 1.89 | 2.24 | | | |
| T41 | 8.51 | 4.29 | 4.15 | 1.23 | | — | — |
| L42 | 8.59 | 4.38 | 1.66 | 1.66 | | 0.93 | — |
| | | | 1.57 | | | 0.87 | |
| R43 | 8.82 | 4.34 | 1.79 | 1.63 | | 3.21 | 7.42 |
| | | | 1.88 | 1.63 | | 3.21 | |
| G44 | 8.68 | 3.95 | — | — | | — | — |
| | | 3.95 | | | | | |
| D45 | 8.36 | 4.60 | 2.70 | — | | — | — |
| | | | 2.68 | | | | |
| E46 | 8.60 | 4.21 | 2.06 | 2.29 | | — | — |
| | | | 1.98 | 2.31 | | | |
| R47 | 8.41 | 4.27 | 1.80 | 1.62 | | 3.19 | 7.40 |
| | | | 1.80 | 1.62 | | 3.19 | |
| I48 | 8.20 | 4.12 | 1.87 | 0.90 | (1a) | 0.85 | — |
| | | | | 1.48 | (1b) | | |
| | | | | 1.18 | (2) | | |
| L49 | 8.41 | 4.37 | 1.64 | 1.64 | | 0.87 | — |
| | | | 1.58 | | | 0.93 | |
| S50 | 8.33 | 4.44 | 3.84 | — | | — | — |
| | | | 3.84 | | | | |
| I51 | 8.16 | 4.19 | 1.88 | 1.18 | (1a) | 0.86 | — |
| | | | | 1.43 | (1b) | | |
| | | | | 0.90 | (2) | | |

| | | | | | | |
|---|---|---|---|---|---|---|
| L52 | 8.36 | 4.39 | 1.62 | 1.62 | 0.93 | — |
| | | | 1.62 | | 0.87 | |
| R53 | 8.00 | 4.17 | 1.84 | 1.58 | 3.17 | 7.24 |
| | | | 1.71 | 1.58 | 3.18 | |

TABLE 6
Chemical shift assignment of 4. Assignment was determined in 20
mM phosphate buffer, 100 NaCl mM, pH 6.5 (10% D₂O) at 280K.

| Residue | HN | Hα | Hβ | Hγ | | Hδ | Hε/HNε |
|---|---|---|---|---|---|---|---|
| F39 | — | 4.29 | 3.15 | — | | 7.26 | 7.38 |
| | | | 3.25 | | | | |
| E40 | 8.76 | 4.45 | 1.90 | 2.25 | | — | — |
| | | | 2.02 | 2.25 | | | |
| T41 | 8.52 | 4.28 | 4.15 | 1.24 | | — | — |
| L42 | 8.61 | 4.38 | 1.57 | 1.66 | | n.a.^{a} | — |
| | | | 1.67 | | | | |
| R43 | 8.55 | 4.33 | 1.79 | 1.64 | | 3.21 | 7.45 |
| | | | 1.89 | 1.67 | | 3.21 | |
| G44 | 8.55 | 3.92 | — | — | | — | — |
| | | 3.92 | | | | | |
| D45 | 8.38 | 4.56 | 2.65 | — | | — | — |
| | | | 2.75 | | | | |
| L46 | 8.29 | 4.27 | 1.61 | 1.67 | | n.a. | — |
| | | | 1.61 | | | | |
| R47 | 8.31 | 4.21 | 1.83 | 1.60 | | 3.21 | 7.48 |
| | | | 1.83 | 1.66 | | 3.21 | |
| I48 | 8.03 | 4.04 | 1.91 | 1.22 | (1a) | 0.87 | — |
| | | | | 1.50 | (1b) | | |
| | | | | 0.92 | (2) | | |
| L49 | 8.26 | 4.27 | 1.57 | 1.68 | | n.a. | — |
| | | | 1.68 | | | | |
| S50 | 8.21 | 4.36 | 3.91 | — | | — | — |
| | | | 3.91 | | | | |
| I51 | 8.06 | 4.06 | 1.92 | 1.17 | (1a) | 0.86 | — |
| | | | | 1.52 | (1b) | | |
| | | | | 0.91 | (2) | | |

| | | | | | | |
|---|---|---|---|---|---|---|
| L52 | 8.20 | 4.26 | 1.55 | 1.68 | n.a. | — |
| | | | 1.68 | | | |
| R53 | 8.28 | 4.24 | 1.81 | 1.57 | 3.18 | 7.29 |
| | | | 1.81 | 1.66 | 3.18 | |
| H54 | 8.42 | 4.60 | 3.23 | — | 7.28 | 8.51 |
| | | | 3.30 | | | |
| Q55 | 8.48 | 4.25 | 2.02 | 2.37 | — | 6.94 |
| | | | 2.06 | 2.37 | | 7.70 |
| N56 | 8.61 | 4.67 | 2.78 | — | 7.03 | — |
| | | | 2.86 | | 7.74 | |
| L57 | 8.29 | 4.30 | 1.62 | 1.67 | n.a. | — |
| | | | 1.62 | | | |
| L58 | 8.15 | 4.29 | 1.58 | 1.68 | n.a. | — |
| | | | 1.68 | | | |
| K59 | 8.16 | 4.25 | 1.79 | 1.43 | 1.69 | 3.00 |
| | | | 1.84 | 1.43 | 1.69 | 3.00 |
| E60 | 8.45 | 4.24 | 1.96 | 2.26 | — | — |
| | | | 2.06 | 2.31 | | |
| L61 | 8.29 | 4.35 | 1.61 | 1.67 | n.a. | — |
| | | | 1.61 | | | |
| Q62 | 8.38 | 4.36 | 1.98 | 2.37 | — | 6.94 |
| | | | 2.15 | 2.37 | | 7.70 |
| D63 | 8.11 | 4.38 | 2.58 | — | — | — |
| | | | 2.67 | | | |

^{a}n.a.: not assigned

TABLE 7
Chemical shift assignment of 5. Assignment
was determined in H₂O (10% D₂O) at 280K.

| Residue | HN | Hα | Hβ | Hγ | | Hδ | Hε/HNε | Hζ |
|---|---|---|---|---|---|---|---|---|
| F39 | 8.39 | 4.53 | 3.01 | — | | 7.25 | — | — |
| | | | 3.12 | | | | | |
| E40 | 8.66 | 4.26 | 2.03 | 2.26 | | — | — | — |
| | | | 1.93 | 2.26 | | | | |
| T41 | 8.20 | 4.26 | 4.21 | 1.22 | | — | — | — |
| L42 | 8.24 | 4.36 | 1.55 | 1.68 | | n.a.^{a} | — | — |
| | | | 1.68 | | | | | |
| R43 | 8.39 | 4.32 | 1.79 | 1.64 | | 3.21 | 7.51 | — |
| | | | 1.89 | 1.64 | | 3.21 | | |
| G44 | 8.53 | 3.96 | — | — | | — | — | — |
| | | 3.89 | | | | | | |
| D45 | 8.45 | 4.54 | 2.70 | — | | — | — | — |
| | | | 2.79 | | | | | |
| L46 | 8.33 | 4.20 | 1.63 | 1.81 | | n.a. | — | — |
| | | | 1.81 | | | | | |
| R47 | 8.21 | 4.13 | 1.95 | 1.59 | | 3.22 | 7.48 | — |
| | | | 1.84 | 1.68 | | 3.22 | | |
| I48 | 7.87 | 3.90 | 1.98 | 1.30 | (1a) | 0.88 | — | — |
| | | | | 1.58 | (1b) | | | |
| | | | | 0.96 | (2) | | | |
| L49 | 8.11 | 4.18 | 1.74 | 1.81 | | n.a. | — | — |
| | | | 1.60 | | | | | |
| S50 | 8.11 | 4.29 | 4.00 | — | | — | — | — |
| | | | 4.05 | | | | | |
| I51 | 8.03 | 3.92 | 2.01 | 1.15 | (1a) | 0.87 | — | — |
| | | | | 1.72 | (1b) | | | |
| | | | | 0.96 | (2) | | | |

| | | | | | | | |
|---|---|---|---|---|---|---|---|
| L52 | 8.15 | 4.15 | n.a. | n.a. | n.a. | — | — |
| R53 | 8.32 | 4.10 | 1.82 | 1.66 | 3.25 | 7.36 | — |
| | | | 1.98 | 1.66 | 3.25 | | |
| X₁54^{b} | 8.26 | 4.54 | 2.76 | — | 7.32 | — | — |
| | | | 2.76 | | | | |
| Q55 | 7.98 | 4.18 | 2.14 | 2.44 | — | 6.91 | — |
| | | | 2.14 | 2.44 | | 7.63 | |
| N56 | 8.14 | 4.46 | 2.87 | — | 6.96 | — | — |
| | | | 2.84 | | 7.71 | | |
| L57 | 7.81 | 4.11 | 1.68 | 1.68 | n.a. | — | — |
| | | | 1.62 | | | | |
| X₂58^{b} | 8.15 | 3.86 | n.a. | n.a. | n.a. | n.a. | n.a. |
| K59 | 7.70 | 4.10 | 1.93 | 1.53 | 1.70 | 2.99 | 7.65 |
| | | | 1.96 | 1.45 | 1.70 | 2.99 | |
| E60 | 7.89 | 4.14 | 2.16 | 2.53 | — | — | — |
| | | | 2.16 | 2.39 | | | |
| L61 | 7.98 | 4.19 | 1.59 | 1.77 | n.a. | — | — |
| | | | 1.77 | | | | |
| Q62 | 8.67 | 3.78 | 2.13 | 2.47 | — | 6.94 | — |
| | | | 2.31 | 2.63 | | 7.63 | |
| D63 | 8.26 | 4.19 | 3.57 | — | — | — | — |
| | | | 3.39 | | | | |

^{a}n.a.: not assigned
^{b}See FIG. 9A for the chemical structure of X₁54and X₂58.

Relaxation experiments on recombinant peptide 1

NMR experiments for the determination of longitudinal and transverse ¹⁵N relaxation rates (R₁=1/T₁ and R₂=1/T₂) and the ¹H-1⁵N heteronuclear NOE (hetNOE)[32] were recorded on ¹³C/1⁵N recombinant peptide 1. Solvent suppression was achieved using pulsed field gradients with a flip-back pulse to avoid saturation of water magnetization which could affect signal intensity of exchangeable amide protons. A series of ¹H-1⁵N-HSQC experiments using different time intervals were recorded for the determination of 1⁵N relaxation rates. T1 measurement, based on inversion-recovery type experiments, were recorded using variable delays 50, 100, 150, 250 (repeated twice for error analysis), 350, 500 (repeated twice for error analysis), 700, 900, 1100, 1400, 2000 ms. T₂ measurement, based on a Carr-Purcell-Meiboom-Gill (CPMG) spin-echo pulse sequence, were acquired using variable delays (8.5 (repeated twice for error analysis), 17, 34, 68 (repeated twice for error analysis), 85, 136, 170, 212.5, 238 ms). T1 and T₂ values were obtained using Dynamics Center Bruker software, by fitting the peak intensity to a 2-parameter exponential decay (FIG. 14). For ¹H-1⁵N heteronuclear NOE measurements, two HSQC spectra were acquired in interleaved fashion with and without 4 s of proton saturation during the relaxation delay. The heteronuclear NOE values were obtained from the ratio between the saturated and unsaturated peak intensities. The uncertainty was calculated as the standard deviation of the noise in the spectrum divided by the intensity of the reference peak. Acquisition parameters of multidimensional experiments are summarized in Table 1.

### Structure calculation

Recombinant peptide 1 structures were calculated with ARIA 2.3.2[33] in combination with CNS using experimentally derived restraints. All NOEs were assigned manually and calibrated by ARIA, the automated assignment was not used. A total of eight iterations was performed, computing 20 structures in the first seven iterations and 300 in the last iteration. The 15 best structures from the last iteration were used for the final default ARIA water refinement step. The quality of the structures was assessed using PROCHECK-NMR software [24]. Statistics of the 15 lowest energy structures are reported in Table 2. The family of the 15 lowest energy structures (no distance or torsional angle restraints violations >0.5 A or >5°, respectively) has been deposited in the PDB (PDB accession code 6R2X). Chemical shift and restraints lists used for structure calculations have been deposited in BioMagResBank (accession code: 34381).

### NMR Binding Experiments

1D ¹H STD and WaterLOGSY experiments

1D ¹H STD measurements (pulse sequence: stddiffesgp.3) were acquired in NMR buffer on peptide 1 (0.5 mM) in the presence of 4 μM recombinant human αvβ6 extracellular domain (R&D Systems) using a pulse scheme with excitation sculpting with gradients for water suppression and spin-lock field to suppress protein signals [34]. The spectra were acquired using 800-4000 scans. For protein saturation, a train of 60 Gaussian shaped pulses of 50 ms was applied, for a total saturation time of 3 s. Relaxation delay was set to 3 s. On-resonance irradiation was set at 12 ppm; off-resonance irradiation was applied at 107 ppm. STD spectra were obtained by internal subtraction of the on-resonance spectrum from the off-resonance spectrum. WaterLOGSY [35] experiments were acquired on the same samples using 256 scan with 20 ppm spectral width, using a tm,ₓ of 1 s and a relaxation delay of 2 s. 2D-STD-HSQC experiments 2D-STD-1H-¹³C-HSQC experiments (stdhsqcetgpsp) were recorded on ¹³C/1⁵N recombinant peptide 1 (0. 5 mM) in NMR buffer (100% D₂O ) the presence of 4 μM recombinant human αvβ6 extracellular domain (R&D Systems), by applying on ¹H on-resonance and off-resonance irradiation at 10.5 ppm and 107 ppm, respectively. Protein saturation was obtained using a train of Gaussian shaped pulses of 50 ms each. A total saturation time of 2.5 s and a relaxation delay of 2.5 s were used, with a time domain of 2048 points in the direct dimension and 160 complex points in the indirect dimension with a total 128 scans. The spectral width was set to 12 ppm for the direct dimension and 80 ppm for carbon dimension. The STD difference was obtained internally by phase cycling. 2D-STD-1H-1⁵N-HSQC experiments [25] were acquired on ¹³C/1⁵N recombinant peptide 1 (0. 5 mM) in NMR buffer containing 10% D₂0 in the presence of 4 ptM recombinant human αvβ6 extracellular domain (R&D Systems). The experiment consists of a 1⁵N HSQC with echo/antiecho coherence selection and water flip back pulses in both inept steps to which a saturation transfer element[34] is prepended. The latter consists of a train of Gaussian shaped pulses of 50 ms, executed multiple times to achieve 3 s of saturation period.

The relaxation delay was set to 3 s. 2048 points were acquired for the direct dimension and 40 complex points on the indirect dimension. In total 224-312 scans were used with a spectral width of 12 and 21 ppm for proton and nitrogen dimensions respectively. The saturation element is applied on- and off-resonance (-3 ppm and 107 ppm, respectively) in alternating scans which are kept in separate blocks of the memory until the chosen number of scans is reached. The data are then stored on the disk and the t1 delay is incremented to obtain the final interleaved 2D. The data are preprocessed using the c-program split (Bruker TopSpin 3.2 software) with the argument 2 to get the off-resonance reference spectrum and with the argument ipap 2 to obtain the difference spectrum in which only signals that did experience saturation transfer are visible. For negative control experiments similar spectra were acquired on labelled recombinant peptide 1 in the absence of αvβ6, in the presence of αvβ6 which was previously incubated with 20 mM EDTA-d16 (Cambridge Isotope Laboratories, Inc), or in the presence of 4 ptM Bovine Serum Albumin (Merck) (FIG. 6). For each non overlapping resonance the relative STD % was evaluated as follows:

$$STD factor = \frac{{I}_{S T D}}{{I}_{ref}} \qquad ( eq . 1 )$$

$$relative STD \% = \frac{STD factor}{STD {factor}_{max}} 100 \qquad ( eq . 2 )$$

where I_{STD} is the peak intensity in the 2D-STD-1H-1⁵N-HSQC spectrum, I_{ref} is the peak intensity of the reference (off-resonance) 2D-STD-1H-1⁵N-HSQC spectrum, and STDfactorₘₐₓ is the maximum value of the STDfactor [25].

### Docking Calculations

HADDOCK2.2 [36,37] was used for the docking calculation of peptides 1, 4 and 5 into αvβ6. As input structures for peptide 1, an ensemble of 10 out of the 30 best NMR structures in terms of energy were acetylated at the N-terminus and amidated at the C-terminus using Maestro (Schrödinger, LLC, New York, N.Y., 2019). Input structures for 4 and 5 were first generated modifying the ensemble of structures of peptide 1 and then minimized using Maestro. Missing topologies and parameters were determined with PRODRG2 web server [38]. The structure of the extracellular head of αvβ6 (P-propeller of the av subunit and pl domain of the P6 subunit) when bound to proTGF-(31 (PDB: 5FFO) [16] was prepared using the Protein Preparation Wizard tool of Maestro [39]. All the crystallographic water molecules were removed. Missing side-chains, hydrogen atoms and loops were added; the orientation of the hydroxyl groups of Serine, Threonine and Tyrosine, the side chains of Asparagine and Glutamine residues, and the protonation state of Histidine residues were optimized. A restrained minimization was run using the OPLS-AA force field [40] with a root mean square deviation (RMSD) tolerance on heavy atoms of 0.3 Å.

In order to maintain a stable coordination of the ions in the metal ion-dependent adhesion site (MIDAS), adjacent to MIDAS (ADMIDAS) and ligand-associated metal binding site (LIMBS), unambiguous restraints were applied throughout the whole docking protocol between Mg² ions and the coordinating residues in the MIDAS (Asp1230₆, Ser1250₆, Ser1270₆, Thr2210₆, Glu2230₆, Asp2540₆), ADMIDAS (Ser1270₆, Asp1300₆, Asp1310₆, Asp2540₆) and LIMBS (Glu1620₆, Asn2180₆, Asp2200₆, Pro2220₆, Glu2230₆). Furthermore, as the binding of CgA to αvβ6 is RGD dependent additional unambiguous restraints were applied during it0 and it1 between: R43 of the input peptides and av residue Asp150av and Asp218ₐv; D45 of the input peptides and the Mg² ion in the MIDAS. For αvβ6, active and passive residues were selected from the 5FFO PDB structure as follows: residues involved in the RGD electrostatic clamp and residues within a radius of 5 Afrom the interacting fragment of TGF-(31 (F210 -P227), with a water accessibility of the main chain and side chain higher than 10%, as determined by Naccess 2.1.1 [41]. For peptide 1, RGD and the residues which gave a relative STD % >75% in the 2D-STD-1H-1⁵N-HSQC experiment, were chosen as active; the remaining residues of the peptide were used as passive. For 4 and 5, G44, D45, L46 residues were chosen as active; the remaining residues of the peptide were used as passive. The list of active and passive residues for the definition of the AIRs is summarized in Table 8.

TABLE 8
List of active and passive residues. Residues selected for the generation of
the Ambiguous Interaction Restraints (AIRs)s for HADDOCK calculations.

| | | Active residues | Passive residues |
|---|---|---|---|
| αvβ6 | αv | Asp150, Asp218 | Asp148, Phe177, Tyr178, Gln180, |
| | subunit | | Ala215 |
| | β6 | MIDAS (Mg²⁺), | Ser127, Asp129, Asp130, Glu175, |
| | subunit | Ala126, Pro179, | Cys180, Ser181, Ser182, Pro184, |
| | | Ile183, Ala217, | Tyr185, Cys187, Ile215, Thr221 |
| | | Asn218 | |
| Peptide | | R43, G44, D45, | F39, E40, T41, L42, E46, R47, S50, |
| 1 | | I48, L49, I51, | R53, H54, Q55, N56, L57, L58, |
| | | L52 | K59, E60, L61, Q62, D63 |
| Peptide | | R43, G44, D45, | F39, E40, T41, L42, E46, R47, I48, |
| 4 | | L46 | L49, S50, I51, L52, R53, H54, Q55, |
| | | | N56, L57, L58, K59, E60, L61, |
| | | | Q62, D63 |
| Peptide | | R43, G44, D45, | F39, E40, T41, L42, E46, R47, I48, |
| 5 | | L46 | L49, S50, I51, L52, R53, X₁54^{a}, |
| | | | Q55, N56, L57, X₂58^{a}, K59, E60, |
| | | | L61, Q62, D63 |

^{a}Stapling residue

The HADDOCK protocol involves three main steps. After the rigid body docking the best 1000 structures in terms of HADDOCK score were then subjected to the semi-flexible refinement step. In this stage, for all peptides, the backbone of residues from F39 to E46 was maintained fully flexible. In this case the best 500 structures, according to the HADDOCK score, were selected for the water refinement stage. Also for the last water refinement stage, residues F39 to E46 were maintained fully flexible. OPLS force field [40] and TIP3P water model [42] were applied. The best 500 decoy poses in terms of HADDOCK score were then clusterized based on geometrical criteria. Poses were aligned on the αvβ6 backbone, and the RMSD was calculated on the backbone of the ligand from R43 to L52 and side chains of the R43 and D45. RMSD cutoff was set to 3.5 A, and only clusters containing more than five structures were considered. To remove any bias of the cluster size on the cluster statistics, the final overall score of each cluster was calculated on the four lowest HADDOCK scores models in each cluster (FIG. 15).

### Circular Dichroism (CD) Spectroscopy

CD spectra were recorded on a Jasco J-815 spectropolarimeter equipped with a Peltier temperature control system. Typical peptides concentration was 30-40 μM, in phosphate buffer 20 mM, NaF 100 mM, pH 6.5. Spectra were acquired in a 1 mm quartz cuvette, at 280 K using an average of four scans between 190 and 260 nm, with a scanning speed of 20 nm/min, 0.5 s of data integration time and a resolution of 0.1 nm.

### Competitive Integrin Binding Assays

Peptide binding was measured by a competitive binding assay using as integrin probe a complex made by a N-terminal acetylated isoDGR peptide biotinylated at the E-amino group of the lysine, acetyl-CisoDGRCGVRSSSRTPSDKY-bio (SEQ ID No. 29), and a streptavidin-peroxidase conjugate (called isoDGR/STV-HRP)[43]. The equilibrium dissociation constants (K_{d}) of the isoDGR/STV-HRP was determined by direct binding assay to 96-well microtiterplates coated with αvβ3, αvβ5, αvβ6, and αvβ8 (1 μg/ml) or with asp₁ (4 μg/ml) and were calculated by non-linear regression analysis using “One site -Specific binding” equation of the GraphPad Prism Software. The following K_{d} values were obtained: αvβ3, 1.3 nM; αvβ5, 1.7 nM; αvβ6, 1.4 nM; αvβ8, 1.6 nM and a5p1, 1.3 nM.

Next, to determine the Ki values for each peptide the inventors performed competitive binding assays using a fixed concentration of the isoDGR/STV-HRP probe (1.68 nM, for αvβ3 and αvβ5; 1.00 nM for αvβ6; 2.00 nM for αvβ8 and 7.12 nM for a5p1) mixed in binding buffer (25 mM Tris-HCl, pH 7.4, containing 150 mM sodium chloride, 1 mM magnesium chloride, 1 mM manganese chloride and 1% BSA) with each competitor at various concentrations (6 dilution in duplicate or triplicates).

Each mixture was then added to integrin-coated wells and left to incubate for 2 h at room temperature. After washing with 25 mM Tris-HCl, pH 7.4, containing 150 mM sodium chloride, 1 mM magnesium chloride, 1 mM manganese chloride, each well was filled with a chromogenic solution (o-phenylenediamine dihydrochloride) and left to incubate for 30 min at room temperature. The chromogenic reaction was stopped by adding 1 N sulfuric acid. The absorbance at 490 nm was then measured using a microtiterplate reader. Kᵢ values were calculated by non-linear regression analysis of competitive binding data using the “One site - Fit Ki” equation of the GraphPad Prism Software using the K_{d} values of the probe indicated above.

### Cell Culture

Human bladder cancer 5637 cells (ATCC HTB-9, grade II carcinoma) were cultured in RPMI-1640 medium, supplemented with 10% heat-inactivated fetal bovine serum (FBS), 2 mM L-glutamine, 100 U/ml penicillin and 100 μg/ml streptomycin. Human skin keratinocytes cells (HaCaT) were kindly provided by Dr. Alessandra Boletta (San Raffaele Scientific Institute, Italy). HaCaT were cultured in DMEM containing 10% FBS, 2 mM L-glutamine, 100 U/ml penicillin and 100 μg/ml streptomycin.

Human BxPC-3 pancreatic adenocarcinoma (ATCC CRL-1687), human 5637 bladder carcinoma, and murine 4T1 mammary carcinoma cells (ATCC CRL-2539) were from ATCC; murine K8484 and DT6606 (pancreatic adenocarcinoma cells were kindly provided by Prof. Lorenzo Piemonti (San Raffaele Scientific Institute, Olive KP, Jacobetz MA, Davidson CJ, Gopinathan A, McIntyre D, Honess D, et al.

Inhibition of Hedgehog signaling enhances delivery of chemotherapy in a mouse model of pancreatic cancer. Science 2009; 324:1457-61 and Celesti G, Di Caro G, Bianchi P, Grizzi F, Marchesi F, Basso G, et al. Early expression of the fractalkine receptor CX3CR1 in pancreatic carcinogenesis. Br J Cancer 2013; 109:2424-33). These cell lines were cultured in RPMI-1640 medium with standard supplements.

Human umbilical vein endothelial cells (HUVEC) were purchased from Lonza and cultured as recommended by the manufacturer. A vial of working cell bank was used to start new experiments; the cells were cultured for not more than 4 weeks before use. All cell lines were mycoplasma-free, as routinely tested using the MycoAlert Control Set (Lonza).

### Flow Cytometry Analysis

Flow cytometry analysis of αvβ6 integrin was carried out as follows: 5637 or HaCaT cells were detached with Dulbecco's Phosphate Buffered Saline (DPBS, without CaCl₂ and MgCl₂) containing 5 mM EDTA pH 8.0 solution (DPBS-E), washed twice with DPBS and resuspended with 25 mM Hepes buffer, pH 7.4, containing 150 mM NaCl, 1 mM MgCl₂, 1 mM MnCl₂ and 1% bovine serum albumin (binding buffer) in presence of various amount of peptides 1, 2, 4, 5 or 6 and mAb 10D5 (5 μg/ml, 33 nM), for 1 h on ice (5×10⁵ cells/100 pl). After washing with 25 mM Hepes buffer, pH 7.4, 150 NaCl, 1 mM MgCl₂, 1 mM MnCl₂, the cells were incubated with a goat anti-mouse Alexa Fluor 488 conjugated secondary antibody (5 μg/ml in binding buffer containing 1% normal goat serum) for 1 h on ice. After washing, the cells were fixed with 4% formaldehyde in DPBS and analyzed by flow cytometry. Flow cytometry analysis of αvβ8 was performed essentially as described above using a rabbit anti-αvβ8 monoclonal antibody (clone EM13309, 1 μg/ml) and a goat anti-rabbit Alexa Flour 488 conjugated secondary antibody (5 μg/ml).

### Preparation of Quantum Dots (Qdot) Labelled with 5

Amine-modified Qdot nanoparticles (2 nmol of Qdot605 ITK Amino (PEG), Invitrogen, Carlsbad, Calif.) were buffer-exchanged in PBS (10 mM sodium phosphate buffer, pH 7.4, 138 mM NaCl, 2.7 mM KCl, Sigma, P-3813) containing 5 mM EDTA (PBS-E) by ultrafiltration using Ultra-4 Ultracel-100K (Amicon) according to the manufacturer's instructions. Qdot were then activated with 200 μg of sulfo-SMCC sulfosuccinimidyl 4-[N-maleimidomethyl] cyclohexane-1-carboxylate (Sulfo-SMCC; Pierce, Rock- ford, IL), a heterobifunctional cross-linking reagent, 1 h at room temperature. The maleimide-tagged nanoparticles were purified from the unreacted cross-linking reagent by gel-filtration chromatography on NAP-5 column (GE Healthcare) using PBS-E as eluent buffer. The product was then divided into 2 aliquots (-300 pl each) and mixed with 5 or a control peptide (cyclic head-to-tail c(CGARAG)) (480 μg in 96 pl of water) and incubated for 2 h at room temperature. 2-mercaptoethanol was then added (0.1 mM final concentration) and left to incubate for 0.5 h at room temperature. Conjugates (called 5-Qdot and *Qdot) were separated from free peptide by ultrafiltration using Ultra-4 Ultracel-100 K, resuspended in 100 mM Tris-HCl, pH 7.4 (300 pl).

The concentrations of 5-Qdot and *Qdot used in the binding assay were determined spectrofluorimetrically using unconjugated Qdot in 25 mM Tris-HCl, 150 mM NaCl, 1 mM MnCl₂, 1 mM MgCl₂ supplemented with 1% BSA as reference standard (1:5 dilution, in triplicate, 200 al/well). The fluorescence of samples and standards were then measured using a Victor Wallac3 instrument (PerkinElmer, excitation filter F355 nm; emission filter 590 nm, bandwidth ±10 nm).

### Binding Assay of 5-Qdot to Human 5637 Cells

Binding assays of 5-Qdot and *Qdot to 5637 cells were carried out as follows: 5637 cells were grown in chamber slides (6×10⁴ cells/well). The cells were washed with 25 mM HEPES buffer, pH 7.4, containing 150 mM NaCl, 1 mM MgCl₂, 1 mM MnCl₂ and incubated with 5-Qdot or *Qdot solution (3.3 nM in binding buffer) for 2 h at 37° C., 5% CO₂. The cells were washed again with binding buffer, fixed with paraformaldehyde for 20 min, counterstained with DAPI (0.05 μg/ml, Invitrogen), and analyzed using a fluorescence microscopy (Carl Zeiss, Axioscop 40FL; excitation, filter, BP 560/40 nm; beam splitter filter, FT 585 nm; emission filter, 630/75 nm). FACS analysis was carried out as follows: the cells were detached with DPBS-E solution, washed with DPBS, resuspended in binding buffer containing 5-Qdot or *Qdot (11-3.7 nM, 5×10⁵ cells/100 pl tube), and left to incubate 2 h at 37° C. After washing with 25 mM Hepes buffer, pH 7.4, containing 150 mM NaCl, 1 mM MgCl₂, 1 mM MgCl₂, the cells were fixed with formaldehyde and analyzed using the CytoFLEX S (Beckman Coulter).

Peptide Stability Assay

Peptide Stability in Human Serum

The stability of 4 and 5 in serum was assessed by ELISA using mAb 5A8, an anti-CgAs₄s₇ antibody (against the sequence HQNL)[44] that can cross-react with 5 (See FIG. 11 for experimental set up).

To this aim, both peptides were synthetized with an additional N-terminal cysteine residue (4a and 5a) (Table 4) to allow coupling to maleimide-activated HRP (Expedeon). 4- and 5-HRP conjugate (called 4-HRP and 5-HRP) were prepared by mixing 24 μg of peptide (5 pl) with 528 μg (108 pl) of maleimide-activated HRP (1:1 ratio) in PBS containing 5 mM EDTA (150 1l final volume) followed by incubation for 3 h at room temperature. To test its stability in serum, 20 0.1l aliquots of 100 nM of peptide-HRP were added to 200 Il aliquots of human serum (Sigma, precleared by centrifugation at 15000 g, 10 min, 4° C.) and incubated at 37° C. Aliquots were collected at different times (0, 1, 2, 4, 8 and 24 h), blocked by adding a solution consisting of Inhibitor Protease Cocktail Ill (1:100, final dilution, Calbiochem) and 10 mM EDTA, pH 8.0 (final concentration). The products were then diluted (1-0.25 nM final concentration) with 50 mM sodium phosphate buffer, pH 7.4, containing 150 mM NaCl and 1% w/v heat denatured BSA, and added to microtiterplates pre-coated with mAb 5A8 (5 μg/ml in 50 mM sodium phosphate buffer, pH 7.4, containing 150 mM NaCl, 50 al/well, overnight at 4° C.). After washing, the peptide-peroxidase conjugate bound to the plate was determined using the o-phenylendiamine chromogenic substrate of HRP. In parallel, the effect of serum on the peroxidase activity of the conjugate was also checked by measuring the enzyme activity in all samples using the same chromogenic substrate.

Peptide Stability in Mouse Liver Microsomal Preparations

The stability of compound 4 and 5 in mouse liver microsomal preparations was assessed by HPLC analysis. To this aim microsomes were prepared as follows: 11 g of mouse liver tissue (C57BL/6 mice) was homogenized in cold PBS supplemented with 0.25 M sucrose (3 ml/g of tissue) using a Potter-Elvehjem homogenizer (10 strokes), followed by additional homogenization using a rotor-stator homogenizer (40 sec). The homogenate was filtered through 70 pm cell-strainers, centrifuged three times to remove insoluble materials (500 x g, 5 min; 3000 x g, 30 min; 110000 x g, 90 min, 4° C.). The clear part of the final pellet (i.e. the microsome fraction) was gently resuspended with cold PBS (14 ml) and centrifuged again. The final pellet was resuspended with 8.25 ml of cold PBS (0.75 ml/g of original tissue), aliquoted and stored at −80° C. Protein concentration was measured by measuring the absorbance at 280 nm with a NanoDrop spectrophotometer (Thermo Scientific).

Peptide stability studies were performed as follows: each peptide (100 μg in 20 pl of water) was added to 200 pl aliquots of the microsomal preparation (2.5 mg/ml protein concentration) and incubated at 37° C. Aliquots were collected at different times (0, 1, 2, 4, 8, 24, 48 and 120 h), diluted with 200 pl of 90% acetonitrile containing 0.1% TFA and stored at −80° C. for subsequent analyses. After thawing, samples were centrifuged (14000 x g, 10 min, 4° C.) and analyzed by HPLC using a C18 LiChrospher column (100 RP-18, 125 mm×4 mm, 5 pm; Merck), as follows: buffer A, 0.1% TFA in water; buffer B, 90% acetonitrile, 0.1% TFA, 0% B (5 min), linear gradient (0-100% B) in 20 min; 100% B (4 min); 0% B, 8 min; flow rate, 0.5 ml/min.

### Images

All 2D structure images reported were prepared using BIOVIA draw (Dassault Systemes BIOVIA, BIOVIA draw, Release 2017, San Diego: Dassault Systemes, 2018).

All 3D structure images were prepared using pymol-1.8.4.2 (The PyMOL Molecular Graphics System, Version 1.8 Schr6dinger, LLC).

Graphs were prepared using Matplotlib,[45] XMGRACE (Turner PJ. XMGRACE, Version 5.1.19. Center for Coastal and Land-Margin Research, Oregon Graduate Institute of Science and Technology, Beaverton, Oreg.; 2005), or Adobe Illustrator 2017.

### Conjugation of Peptides to IRDye800 Fluorophore

Peptide 5a, 2a or cysteine (Cys) were coupled to IRDye 800CW near-infrared fluorescent dye (LI-COR, Lincoln, Nebr., USA) as follows: 60 Il of maleimide-IRDye 800CW (511 nmol) in 10 mM phosphate buffer, pH 7.4, containing 138 mM sodium chloride, 2.7 mM potassium chloride (called PBS-SIGMA) were added to tubes containing 480 Il of 5a, 2a or a Cys (614 nmol) in PBS-SIGMA (dye/peptide ratio,1:1.2) and left to react for 16 h at 4° C. Each product was diluted 2-fold with water and gel-filtered through a Superdex peptide column (10/300 GL, GE Healthcare) pre-equilibrated with 50 mM sodium phosphate buffer, pH 7.4, containing 150 mM sodium chloride (PBS) (flow rate, 0.5 ml/min). The identity of purified products, called 5a-IRDye, 2a-IRDye and Cys-IRDye, were confirmed by mass spectrometry analysis (Table 1). The concentration of each conjugate was quantified by spectrophotometric analysis at 774 nm (molar absorption coefficient, 240,000 M-1, cm-1).

### Binding of Peptide-IRDye Conjugates to αvβ6 and αvβ8 Integrins

Binding of 5a-IRDye, 2a-IRDye and Cys-IRDye to αvβ6 and αvβ8 was determined by direct binding assays as follows: 96-well clear-bottom black microtiterplates (Corning® cat. 3601) were coated with 4 μg/ml αvβ6 or αvβ8, in Dulbecco's phosphate-buffered saline with Ca2+ and Mg2+(DPBS, 50 ul/well) and left to incubate over-night at 4° C. After washing, the plates were incubated with 3% BSA in DPBS (200 al/well, 1 h, room temperature), washed again with 0.9% sodium chloride solution, and filled with various amounts of peptide-IRDye conjugates (range 0.01-100 nM) in 25 mM Tris-HCl, pH 7.4, containing 150 mM sodium chloride, 1 mM magnesium chloride, 1 mM manganese chloride, 0.05% Tween-20 and 1% BSA, 100 pl/well. After 1 h of incubation, the plates were washed three times with the same buffer, without BSA. The bound fluorescence was then quantified by scanning the empty plates with an Odyssey CLx near-infrared fluorescence imaging system (LI-COR) using the following settings: scan intensity, 8.5; scan focus offset, 3; scan quality, highest; channel, 800; resolution, 169 pm.

Binding of Peptide-IRDye Conjugates to Cultured Living Cells

The binding of peptide-IRDye conjugates to BxPC-3, 5637, HUVECs, 4T1, K8484 and DT6606 cells was analyzed as follows. The cells were grown in 96-well clear-bottom black microtiterplates (Corning® cat. 3603, 2-3x104 cells/well, plated 48 h before the experiment). After washing twice with 0.9% sodium chloride solution, the cells were incubated with 25 mM Hepes buffer, pH 7.4, containing 150 mM sodium chloride, 1 mM magnesium chloride, 1 mM manganese chloride and 1% BSA (binding buffer) for 5 min. Peptide-IRDye conjugates (0.013-40 nM in binding buffer) were then added to the cells and left to incubate for 1 h at 37° C., 5% CO2. After three washings with binding buffer (5 min each, 200 al/well), the cells were fixed with PBS containing 2% paraformaldehyde and 3% sucrose for 15 min at room temperature. Binding of conjugates to cells was quantified by scanning the plate (filled with PBS, 100 al/well) with the Odyssey CLx (LI-COR) using the following settings: scan intensity, 8.5; scan focus offset 4; scan quality, highest; channel, 800; resolution, 169 pm. Then, the plates were incubated with 5 μg/ml of 4′,6-diamidino-2-phenylindole (DAPI) for 15 min, washed twice with PBS and read with a VICTOR3 plate reader (Perkin Elmer, Waltham, Mass., USA) to quantify the cell number in each well, using the following filters: excitation, 355±40 nm; emission, 460±25 nm (acquisition,1 s).

### Flow Cytometry Analysis

Flow cytometry analysis of integrins expressed on the surface of cells were carried out using the following antibodies: mouse anti-human/mouse aVP6 antibody (clone 10D5, IgG2a, Millipore); rabbit anti-human αvβ8 antibody (clone EM13309, IgG, Absolute Antibodies); control isotype-matched murine IgG1 (clone MOPC 31C, Sigma); affinity purified (protein-A Sepharose) normal rabbit IgGs (Primm, Italy). The binding of primary antibodies to integrins was detected using goat anti-mouse, or goat anti-rabbit Alexa Fluor 488-labeled secondary antibodies (Invitrogen).

### In Vivo Optical Imaging of BxPC-3 Tumors

All animal procedures were approved by the Ospedale San Raffaele Animal Care and Use Committee (IACUC) and approved by the Istituto Superiore di Sanith of Italy. Eight-weeks old female NSG mice (Charles River Laboratories) were inoculated subcutaneously with 1x107 BxPC-3 cells on the right shoulder. When the tumors reached a diameter of approximately 0.5 cm (0.4-0.6 g, 30-35 days after cell inoculation) mice were anesthetized with 2% isoflurane and subjected to epi-fluorescence imaging before and after intravenous injection of 5a-IRDye (1.28 nmol in 100 pl of 0.9% sodium chloride), or 0.9% sodium chloride. Mice were imaged after 0, 1, 3, and 24 h using the IVIS SpectrumCT imaging system (PerkinElmer) equipped with 745 nm excitation and 800 nm emission filters and the following instrumental settings: exposure, auto; binning, 8; F/stop, 2; field of view, D. Each image acquisition took less than 1 min; images were obtained with four mice at a time. After the final scan, mice were killed and tumor, liver, kidney, spleen, brain, intestine, stomach, pancreas and heart were excised for ex-vivo imaging. Regions of interest (ROI) were drawn on images and the average radiant efficiency was calculated using the Living Image 4.3.1 software (PerkinElmer).

Peptide conjugation to NOTA Peptides 5a and 2a were coupled to maleimide-NOTA (1,4,7-triazacyclononane-1,4-bis-acetic acid-7-maleimidoethylacetamide, CAS number: 1295584-83-6) as follows: 6.5 pmol of maleimide-NOTA (CheMatech, Dijon, France) in 0.445 ml of PBS (SIGMA) was mixed with 3.21 pmol of peptide in 1.555 ml of PBS (NOTA/peptide molar ratio, 2:1) and left to react for 16 h at 4° C., and mixed with 50% (vol/vol) orthophosphoric acid (100 pl). The conjugates were then purified using a semi-preparative reverse-phase HPLC C18 column (LUNA, 250×10 mm, 100 angstrom, 10 pm, Phenomenex) connected to an AKTA Purifier 10 HPLC (GE Healthcare), as follows: mobile phase A, 0.1% trifluoroacetic acid (TFA) in water; mobile phase B, 0.1% TFA in 95% acetonitrile; 0% B (9 min), linear gradient 0-100% B (40 min), 100% B (10 min), 0% B (10 min); flow rate, 5 ml/min. Fractions containing the conjugates were pooled, lyophilized, resuspended in water, and analyzed by analytical reverse-phase HPLC using a C18 column (LUNA, 250×4.6 mm, 100 angstrom, 5 pm, Phenomenex), using the same method as described above except that flow rate was 0.5 ml/min. Product identity was assessed by mass spectrometry analysis (LTQ-XL Orbitrap). ¹⁸F radiolabeling of peptide-NOTA conjugates The peptide 5a-NOTA conjugate was radiolabeled in house with 1⁸F using a modified Tracerlab FX-N automatic module (GE Healthcare, Illinois, USA). 2-4 GBq 18F-sodium fluoride, produced using a IBA 18/9 MeV Cyclotron (IBA RadioPharma Solutions, Louvain-la-Neuve, Belgium), was delivered to the module and loaded onto an anion-exchanger cartridge (Sep-Pak Accell Plus QMA Plus Light, Waters, Italy) pre-conditioned with 0.5 M sodium acetate, pH 8.5 (10 ml), and washed with 10 ml of metal-free water. The column was washed with water (10 ml) and 0.9% sodium chloride (200 pl) and eluted with 0.9% sodium chloride (300 pl). Fluorine was collected into a glassy carbon reactor and mixed with metal-free 0.5 M sodium acetate, pH 4.2 (15 pl), 8.6 mg/ml 5a-NOTA conjugate in water (15 pl), 2 mM aluminum chloride in 0.5 M sodium acetate, pH 4.2 (3.6 pl), 50 mM ascorbic acid in 0.5 M sodium acetate, pH 4.2 (5 pl) and pure ethanol (330 pl). Finally, the product was incubated at 107° C. for 15 min.

After cooling, the product was brought to 10 ml with deionized water, loaded onto a C18 cartridge (Sep-Pak Plus Waters), washed with water (12 ml) and eluted with ethanol/water (1:1) (1 ml). The product was diluted to 5 ml with 0.9% sodium chloride. The radiochemical purity was checked by reverse-phase-HPLC using a C18 column (ACE C18, 250×4.6 mm, Phenomenex) connected to an HPLC system (Waters Corporation, Milford Mass., USA) equipped with a radiochemical counter, using the following chromatographic conditions: buffer A, 0.1% TFA in water; buffer B, 0.1% TFA in acetonitrile; flow, 1 ml/min; linear gradient 0-20 min: 20% B; 20-40 min: 85% B; 40-45 min: 85% B; 45-55 min: 0% B.

### PET Imaging BxPC-3 Tumor-Bearing Mice and Biodistribution Studies

For kinetics studies, the [18F]-NOTA-5a conjugate was injected into the tail vein of BxPC-3 tumor-bearing mice 30-35 days after tumor cell implantation (˜ 4 MBq/mice, in 100 pl of water containing <10% ethanol). The uptake of the radiotracer was monitored by whole-body PET/CT scans using the preclinical p-cube® and X-cube® scanners (Molecubes, Gent, BE), respectively. Three mice were placed side-by-side in a prone position under anesthesia (2% isoflurane in medical air) and imaged after 1, 2, and 4 h. For blocking study, tumor-bearing mice (n=3) were intravenously injected with unlabeled 5a peptide (400 ag/mice, in PBS containing 100 μg/ml human serum albumin), 10 min before the administration of [1⁸F]-NOTA-5a (˜ 3 MBq/mice). After 2 h, mice were whole-body PET/CT imaged and sacrificed for ex-vivo quantification of radiotracer uptake. For ex vivo biodistribution the animals were euthanized by cervical dislocation. Tumor and selected organs were collected, rinsed, weighted and analyzed for their radioactivity content using a y-counter (LKB Compugamma CS 1282). CT and PET images were reconstructed using the proprietary Molecubes software included in the system. CT images were reconstructed with a 200 pm isotropic pixel size using a standard ISRA algorithm. PET images were reconstructed using a List-Mode OSEM algorithm with 30 iterations and 400 pm isotropic voxel size, accounting for the tracer decay correction. CT/PET images were processed by Region of Interest (ROI) analysis using PMOD software v.4.1 (Zurich, Switzerland). The uptake of radioactivity is expressed as “maximum standardized uptake” value (SUV max) and “mean standardized uptake” value (SUV mean), in kinetics and blocking studies respectively. Results and images are also reported as tumor-to-muscle ratio (T/M).

### RESULTS

### Preparation and Characterization of Peptide-IRDye 800CW Conjugates

The CgA39-63-derived peptide (called peptide 5) containing an RGD motif followed by a stapled alpha-helix, is capable of recognizing αvβ6 and αvβ8 integrin with high affinity and selectivity (Table 4).

Furthermore, the non-stapled control peptide with RGE in place of RGD, called peptide 2, is unable to bind these integrins. To couple these peptides to maleimide-IRDye 800CW, a near infrared dye, the inventors fused a cysteine residue to their N-terminus (FIG. 19 and Table 4). Competitive αvβ6 integrin binding assays with these peptides (called 5a and 2a, respectively) showed that their αvβ6 recognition properties were similar to those of 5 and 2, respectively) (Tables 3a and 3b and FIG. 20).

Thus, the fusion of a Cys to 5a N-terminus did not impair its capability to bind αvβ6.

The compounds 5a and 2a were then coupled, via Cys, to maleimide-IRDye 800CW. As a control, a Cys-IRDye 800CW conjugate was also prepared using Cys in place of peptides. The identity of each product (called 5a-IRDye, 2a-IRDye and Cys-IRDye) was checked by MS analysis. Integrin binding assays showed that 5a-IRDye, but not 2a-IRDye or Cys-IRDye, could bind microtiter plates coated with purified Ov06 or Ov08, with an EC50 of 2-3 nM (FIG. 21). These data suggest that 5a maintains its capability to bind both integrins after labelling with IRDye 800CW.

### 5a-IRDye, but not 2a-IRDye or Cys-IRDye, Binds to αvβ6 Positive Cells

To assess the capability of peptide-IRDye conjugates to recognize αvβ6 and αvβ8 also when expressed on cell membranes, the inventors then analyzed the interaction of 5a-, 2a- or Cys-IRDye with αvβ6/αvβ8-positive and -negative cells. To this aim the inventors first characterized, by FACS analysis with specific antibodies, the expression of these integrins by various cell lines, including human BxPC-3 pancreatic adenocarcinoma cells, human 5637 bladder carcinoma cells, murine 4T1 mammary carcinoma cells, and murine K8484 and DT6606 pancreatic adenocarcinoma cells. The results showed that: a) BxPC-3 are αvβ6+ and αvβ8-; b) 5637 cells are αvβ6+ and αvβ8+; c) 4T1 cells are αvβ6+; d) K8484 are αvβ6low and e) DT6606 are αvβ6- (FIG. 22A). The expression of αvβ8 on murine cells was not investigated because the specific antibodies necessary for the FACS analysis of murine P8-positive cells are not available. Interestingly, 5a-IRDye, but not 2a-IRDye or Cys-IRDye, could bind, in a dose dependent manner, cultured αvβ6+cells (e.g. BxPC-3 or 5637), but little or not to cells lacking these integrins (e.g. HUVEC, FIG. 22B). On note, the binding of 5a-IRDye to these cells correlated with the levels of αvβ6 expression.

The binding of 5a-IRDye to BxPC-3 and 5637 cells was significantly inhibited by an excess of free 5a, but not by 2a (FIG. 22C). Notably, the inhibitory potency of 5a was comparable to that of the foot and mouth disease virus-derived peptide A20FMDV2 (peptide 6; Ki, 0.9 nM) (Table 4), i.e. a well-known ligand of αvβ6 (FIG. 22C). These results suggest 5a-IRDye can bind αvβ6 in vitro, either in a purified form (bound to microtiterplates) or when expressed on the cell surface. Furthermore, the lower binding properties of 2a-IRDye (lacking RGD), indicate that the RGD sequence of 5a-IRDye is crucial for binding.

### Imaging of Subcutaneous BxPC-3 Tumors with 5a-IRDye

To assess the capability of 5a-IRDye to bind αvβ6 in vivo the inventors then analyzed the uptake of this conjugate by BxPC-3 tumors (αvβ6+) implanted, subcutaneously, in mice. Maximal uptake was observed after 1 h from injection (FIG. 23A). Twenty-four hours later, a significant signal was still visible (FIG. 23A). At this time, ex vivo NIR-fluorescence measurements showed high accumulation of the conjugate in tumors and kidneys, compared the other organs (FIG. 23B). The high level of kidney fluorescence is in keeping with reported renal clearance of IRDye 800CW (Marshall MV, Draney D, Sevick-Muraca EM, Olive DM. Single-dose intravenous toxicity study of IRDye 800CW in Sprague-Dawley rats. Mol Imaging Biol 2010; 12:583-94). Of note, about three-fold lower accumulation was observed in lung or liver compared to tumor, whereas little or no accumulation was observed in heart, brain, spleen, intestine, stomach or pancreas (FIG. 23B). Interestingly the tumor-to-pancreas ratio was ˜12, suggesting that 5a-IRDye could be exploit for pancreatic tumor imaging.

### Radio-Imaging of Subcutaneous BxPC-3 Tumors with ¹⁸F-NOTA-5a

To further assess the capability of 5a to recognize αvβ6 in in vivo and to accumulate in αvβ6-positive tumors, the inventors coupled this peptide with maleimide-NOTA and labeled the resulting conjugate (NOTA-5a) with 1⁸F (FIG. 24). In parallel, the NOTA-2a conjugate, a negative control, was also prepared. Reverse-phase HPLC and mass spectrometry analysis of NOTA-5a and NOTA-2a showed that both products were homogeneous and characterized by molecular weights consistent with the expected values (FIG. 24A and B). The labeled product, called 1⁸F-NOTA-5a, showed a radiochemical purity >96% and specific activity of 2.2-13.9 MBq/nmole. Moreover, 1⁸F-NOTA-5a showed good stability until 4 h post-production, when stored at room temperature (FIG. 24C).

Furthermore, competitive αvβ6 binding assays showed that NOTA-5a, but not NOTA-2a, can bind αvβ6 with a potency similar to that of the unlabeled 5a peptide (FIG. 24D).

The tumor uptake of 1⁸F-NOTA-5a was then investigated using the subcutaneous BxPC-3 model. Whole body PET/CT scan of tumor-bearing mice, performed 1, 2, and 4 h after 1⁸F-NOTA-5a administration, showed that the radiotracer could accumulate in tumors and kidneys, but not in muscles or femurs, i.e. tissues that do not express αvβ6 or αvβ8 integrin (FIG. 25A). The uptake in kidneys was presumably related to renal clearance, as also suggested by the observation that urine contained high levels of radioactivity (not shown). Notably, the high and progressive accumulation of radiotracer in tumors, but not (or much less in femurs) (FIG. 25B) point to a specific mechanism of uptake. Accordingly, the uptake of 1⁸F-NOTA-5a, 2 h post-injection, was almost completely inhibited by pre-administration of an excess of unlabeled 5a (FIG. 26), lending support to the hypothesis that the radiotracer uptake by tumors was mediated by specific mechanism involving ligand-receptor interactions.

Biodistribution data (performed 2 h post-injection on selected organs) confirmed that the radiotracer accumulated in tumors in a specific manner, as indicated by the marked drop of tumor uptake (from 3.5% to less than 0.5% of the injected dose (ID)/g of tissue) in mice pre-treated with an excess of unlabeled peptide 5a (FIG. 27). Lower, albeit specific, accumulation of 1⁸F-NOTA-5a was observed also in the lungs (1.2% ID/g). The uptake in brain, heart, spleen, blood, muscle was less than 0.5% ID/g and not competed by free peptide. Furthermore, a certain degree of accumulation (about 2% of ID/g) was observed also in intestine, femur, liver and stomach, but also in this case no significant reduction was caused by the unlabeled 5a, arguing against a peptide-mediated mechanism of accumulations in these organs. Finally, high radiotracer levels were also observed in the kidneys (about 80% ID/g), which is likely related to renal clearance of the conjugate.

The references cited throughout this application are incorporated for all purposes apparent herein and in the references themselves as if each reference was fully set forth. For the sake of presentation, specific ones of these references are cited at particular locations herein. A citation of a reference at a particular location indicates a manner(s) in which the teachings of the reference are incorporated.

However, a citation of a reference at a particular location does not limit the manner in which all of the teachings of the cited reference are incorporated for all purposes.

It is understood, therefore, that this invention is not limited to the particular embodiments disclosed, but is intended to cover all modifications which are within the spirit and scope of the invention as defined by the appended claims; the above description; and/or shown in the attached drawings.

### REFERENCES

[1] K.P. Conroy, L.J. Kitto, N.C. Henderson, av integrins: key regulators of tissue fibrosis, Cell Tissue Res. 365 (2016) 511-519. doi:10.1007/s00441-016-2407-9.
[2] L. Koivisto, J. Bi, L. Hakkinen, H. Larjava, Integrin αvβ6: Structure, function and role in health and disease, Int. J. Biochem. Cell Biol. 99 (2018) 186-196. doi:10.1016/j.biocel.2018.04.013.
[3] M. Nieberler, U. Reuning, F. Reichart, J. Notni, H. Wester, M. Schwaiger, M. Weinmuller, A. Rader, K. Steiger, H. Kessler, Exploring the Role of RGD-Recognizing Integrins in Cancer, Cancers (Basel). 9 (2017) 116. doi:10.3390/cancers9090116.
[4] I.D. Campbell, M.J. Humphries, Integrin structure, activation, and interactions, Cold Spring Harb. Perspect. Biol. 3 (2011) 1-14. doi:10.1101/cshperspect.a004994.
[5] A. Ozawa, Y. Sato, T. Imabayashi, T. Uemura, J. Takagi, K. Sekiguchi, Molecular Basis of the Ligand Binding Specificity of αvβ8 Integrin, J. Biol. Chem. 291 (2016) 11551-11565. doi:10.1074/jbc.M116.719138.
[6] F. Reichart, O. V. Maltsev, T.G. Kapp, A.F.B. Rader, M. Weinmuller, U.K. Marelli, J. Notni, A.

Wurzer, R. Beck, H.-J. Wester, K. Steiger, S. Di Maro, F.S. Di Leva, L. Marinelli, M. Nieberler, U. Reuning, M. Schwaiger, H. Kessler, Selective Targeting of Integrin αvβ8 by a Highly Active Cyclic Peptide, J. Med. Chem. 62 (2019) 2024-2037. doi:10.1021/acs.jmedchem.8b01588.
[7] O. V Maltsev, U.K. Marelli, T.G. Kapp, F. Saverio, D. Leva, S. Di Maro, M. Nieberler, U. Reuning, M. Schwaiger, E. Novellino, L. Marinelli, H. Kessler, Supporting Information Selective Integrin Ligands, (n.d.).
[8] A. Cormier, M.G. Campbell, S. Ito, S. Wu, J. Lou, J. Marks, J.L. Baron, S.L. Nishimura, Y. Cheng, Cryo-EM structure of the αvβ8 integrin reveals a mechanism for stabilizing integrin extension, Nat. Struct. Mol. Biol. 25 (2018) 698-704. doi:10.1038/s41594-018-0093-x.
[9] F.S. DiLeva, S. Tomassi, S. DiMaro, F. Reichart, J. Notni, A. Dangi, U.K. Marelli, D. Brancaccio, F.

Merlino, H.J. Wester, E. Novellino, H. Kessler, L. Marinelli, From a Helix to a Small Cycle: Metadynamics-Inspired αvβ6 Integrin Selective Ligands, Angew. Chemie - Int. Ed. 44 (2018) 4645-14649. doi:10.1002/anie.201803250.
[10] M. Civera, D. Arosio, F. Bonato, L. Manzoni, L. Pignataro, S. Zanella, C. Gennari, U. Piarulli, L.

Belvisi, Investigating the Interaction of Cyclic RGD Peptidomimetics with aVP6 Integrin by Biochemical and Molecular Docking Studies, Cancers (Basel). 9 (2017) 128. doi:10.3390/cancers9100128.
[11] S.H. Hausner, D. DiCara, J. Marik, J.F. Marshall, J.L. Sutcliffe, Use of a peptide derived from foot-and-mouth disease virus for the noninvasive imaging of human cancer: Generation and evaluation of 4-[18F]fluorobenzoyl A20FMDV2 for in vivo imaging of integrin αvβ6expression with positron emission tomography, Cancer Res. 67 (2007) 7833-7840. doi:10.1016/j.jallcom.2012.12.082.
[12] R.H. Kimura, R. Teed, B.J. Hackel, M.A. Pysz, C.Z. Chuang, A. Sathirachinda, J.K. Willmann, S.S.

Gambhir, Pharmacokinetically Stabilized Cystine Knot Peptides That Bind Alpha-v-Beta-6 Integrin with Single-Digit Nanomolar Affinities for Detection of Pancreatic Cancer, Clin. Cancer Res. 18 (2012) 839-849. doi:10.1158/1078-0432.CCR-11-1116.
[13] A. Altmann, M. Sauter, S. Roesch, W. Mier, R. Warta, J. Debus, G. Dyckhoff, C. Herold-Mende, U.

Haberkorn, Identification of a Novel ITGαvβ6-Binding Peptide Using Protein Separation and Phage Display., Clin. Cancer Res. 23 (2017) 4170-4180. doi:10.1158/1078-0432.CCR-16-3217.
[14] S. Kraft, B. Diefenbach, R. Mehta, A. Jonczyk, G.A. Luckenbach, S.L. Goodman, Definition of an unexpected ligand recognition motif for αvβ6 integrin, J. Biol. Chem. 274 (1999) 1979-1985. doi:10.1074/jbc.274.4.1979.
[15] X. Dong, N.E. Hudson, C. Lu, T.A. Springer, Structural determinants of integrin -subunit specificity for latent TGF-P, Nat. Struct. Mol. Biol. 21 (2014) 1091-1096. doi:10.1038/nsmb.2905.
[16] X. Dong, B. Zhao, R.E. lacob, J. Zhu, A.C. Koksal, C. Lu, J.R. Engen, T.A. Springer, Force interacts with macromolecular structure in activation of TGF-P, Nature. 542 (2017) 55-59. doi:10.1038/nature21035.
[17] A. Kotecha, Q. Wang, X. Dong, S.L. Ilca, M. Ondiviela, R. Zihe, J. Seago, B. Charleston, E.E. Fry, N.G.A. Abrescia, T.A. Springer, J.T. Huiskonen, D.I. Stuart, Rules of engagement between αvβ6 integrin and foot-and-mouth disease virus, Nat. Commun. 8 (2017) 15408-15416. doi:10.1038/ncomms15408.
[18] D. DiCara, C. Rapisarda, J.L. Sutcliffe, S.M. Violette, P.H. Weinreb, I.R. Hart, M.J. Howard, J.F. Marshall, Structure-Function Analysis of Arg-Gly-Asp Helix Motifs in αvβ6 Integrin Ligands, J.

Biol. Chem. 282 (2007) 9657-9665. doi:10.1074/jbc.M610461200.
[19] A. Corti, F. Marcucci, T. Bachetti, Circulating chromogranin A and its fragments as diagnostic and prognostic disease markers, Pflugers Arch. Eur. J. Physiol. 470 (2018) 199-210. doi:10.1007/s00424-017-2030-y.
[20] K.B. Helle, M.H. Metz-Boutigue, M.C. Cerra, T. Angelone, Chromogranins: from discovery to current times, Pflugers Arch. Eur. J. Physiol. 470 (2018) 143-154. doi:10.1007/s00424-017-2027-50 6.
[21] F. Curnis, A.M. Gasparri, R. Longhi, B. Colombo, S. D′Alessio, F. Pastorino, M. Ponzoni, A. Corti, Chromogranin A binds to αvβ6-integrin and promotes wound healing in mice, Cell. Mol. Life Sci. 69 (2012) 2791-2803. doi:10.1007/s00018-012-0955-z.
[22] M.A. Larkin, G. Blackshields, N.P. Brown, R. Chenna, P.A. Mcgettigan, H. McWilliam, F. Valentin, I.M. Wallace, A. Wilm, R. Lopez, J.D. Thompson, T.J. Gibson, D.G. Higgins, Clustal W and Clustal X version 2.0, Bioinformatics. (2007). doi:10.1093/bioinformatics/btm404.
[23] X. Robert, P. Gouet, Deciphering key features in protein structures with the new ENDscript server, Nucleic Acids Res. 42 (2014) W320-W324. doi:10.1093/nar/gku316.
[24] R.A. Laskowski, J.A. Rullmannn, M.W. MacArthur, R. Kaptein, J.M. Thornton, AQUA and PROCHECK-NMR: programs for checking the quality of protein structures solved by NMR., J.

Biomol. NMR. 8 (1996) 477-86. doi:10.1007/BF00228148.
[25] J.L. Wagstaff, S. Vallath, J.F. Marshall, R.A. Williamson, M.J. Howard, Two-dimensional heteronuclear saturation transfer difference NMR reveals detailed integrin αvβ6 protein-peptide interactions, Chem. Commun. 46 (2010) 7533. doi:10.1039/c0cc01846e.
[26] Y.K.S. Man, D. DiCara, N. Chan, S. Vessillier, S.J. Mather, M.L. Rowe, M.J. Howard, J.F. Marshall, A. Nissim, Structural Guided Scaffold Phage Display Libraries as a Source of Bio-Therapeutics, PLoS One. 8 (2013) e70452. doi:10.1371/journal.pone.0070452.
[27] Y.S. Tan, D.P. Lane, C.S. Verma, Stapled peptide design: principles and roles of computation, Drug Discov. Today. 21 (2016) 1642-1653. doi:10.1016/j.drudis.2016.06.012.
[28] A. Gori, C.-l.A. Wang, P.J. Harvey, K.J. Rosengren, R.F. Bhola, M.L. Gelmi, R. Longhi, M.J. Christie, R.J. Lewis, P.F. Alewood, A. Brust, Stabilization of the Cysteine-Rich Conotoxin MrIA by Using a 1,2,3-Triazole as a Disulfide Bond Mimetic, Angew. Chemie Int. Ed. 54 (2015) 1361-1364. doi:10.1002/anie.201409678.
[29] T.G. Kapp, F. Rechenmacher, S. Neubauer, O. V. Maltsev, E.A. Cavalcanti-Adam, R. Zarka, U.

Reuning, J. Notni, H.J. Wester, C. Mas-Moruno, J. Spatz, B. Geiger, H. Kessler, A comprehensive evaluation of the activity and selectivity profile of ligands for RGD-binding integrins, Sci. Rep. 7 (2017) 1-12. doi:10.1038/srep39805.
[30] X. Huang, J. Wu, S. Spong, D. Sheppard, The integrin αvβ6 is critical for keratinocyte migration on both its known ligand, fibronectin, and on vitronectin., J. Cell Sci. 111 (1998) 2189-2195.
[31] W.F. Vranken, W. Boucher, T.J. Stevens, R.H. Fogh, A. Pajon, M. Llinas, E.L. Ulrich, J.L. Markley, J. lonides, E.D. Laue, The CCPN data model for NMR spectroscopy: Development of a software pipeline, Proteins Struct. Funct. Genet. 59 (2005) 687-696. doi:10.1002/prot.20449.
[32] N.A. Farrow, R. Muhandiram, A.U. Singer, S.M. Pascal, C.M. Kay, G. Gish, S.E. Shoelson, T.

Pawson, J.D. Forman-Kay, L.E. Kay, Backbone Dynamics of a Free and a Phosphopeptide-Complexed Src Homology 2 Domain Studied by 15N NMR Relaxation, Biochemistry. 33 (2005) 5984-6003. doi:10.1021/bi00185a040.
[33] W. Rieping, B. Bardiaux, A. Bernard, T.E. Malliavin, M. Nilges, ARIA2: Automated NOE assignment and data integration in NMR structure calculation, Bioinformatics. 23 (2007) 381-382. doi:10.1093/bioinformatics/btl589.
[34] M. Mayer, B. Meyer, Characterization of ligand binding by saturation transfer difference NMR spectroscopy, Angew. Chemie - Int. Ed. 38 (1999) 1784-1788. doi:10.1002/(SICI)1521-3773(19990614)38:12<1784::AID-ANIE1784>3.0.CO;2-Q.
[35] C. Dalvit, P. Pevarello, M. Tato, M. Veronesi, A. Vulpetti, M. Sundstr??m, Identification of compounds with binding affinity to proteins via magnetization transfer from bulk water, J. Biomol. NMR. 18 (2000) 65-68. doi:10.1023/A:1008354229396.
[36] C. Dominguez, R. Boelens, A.M.J.J. Bonvin, HADDOCK: A protein-protein docking approach based on biochemical or biophysical information, J. Am. Chem. Soc. 125 (2003) 1731-1737. doi:10.1021/ja026939x.
[37] S.J. de Vries, A.D.J. van Dijk, M. Krzeminski, M. van Dijk, A. Thureau, V. Hsu, T. Wassenaar, A.M.J.J. Bonvin, HADDOCK versus HADDOCK: New features and performance of HADDOCK2.0 on the CAPRI targets, Proteins Struct. Funct. Bioinforma. 69 (2007) 726-733. doi:10.1002/prot.21723.
[38] A.W. Schuttelkopf, D.M.F. van Aalten, PRODRG: a tool for high-throughput crystallography of protein-ligand complexes, Acta Crystallogr. Sect. D Biol. Crystallogr. 60 (2004) 1355-1363. doi:10.1107/S0907444904011679.
[39] G. Madhavi Sastry, M. Adzhigirey, T. Day, R. Annabhimoju, W. Sherman, Protein and ligand preparation: Parameters, protocols, and influence on virtual screening enrichments, J. Comput. Aided. Mol. Des. 27 (2013) 221-234. doi:10.1007/s10822-013-9644-8.
[40] G.A. Kaminski, R.A. Friesner, J. Tirado-Rives, W.L. Jorgensen, Evaluation and reparametrization of the OPLS-AA force field for proteins via comparison with accurate quantum chemical calculations on peptides, J. Phys. Chem. B. 105 (2001) 6474-6487. doi:10.1021/jp003919d.
[41] Hubbard, J. Thornton, “NACCESS”, computer program., Comput. Progr. (1993) http://wolf.bms.umist.ac.uk/naccess/.
[42] W.L. Jorgensen, J. Chandrasekhar, J.D. Madura, R.W. Impey, M.L. Klein, Comparison of simple potential functions for simulating liquid water, J. Chem. Phys. 79 (1983) 926-935. doi:10.1063/1.445869.
[43] F. Curnis, A.M. Gasparri, R. Longhi, B. Colombo, S. D′Alessio, F. Pastorino, M. Ponzoni, A. Corti, Chromogranin A binds to αvβ6-integrin and promotes wound healing in mice, Cell. Mol. Life Sci. 69 (2012) 2791-2803. doi:10.3966/160792642018051903020.
[44] B. Colombo, R. Longhi, C. Marinzi, F. Magni, A. Cattaneo, S.H. Yoo, F. Curnis, A. Corti, Cleavage of Chromogranin A N-terminal Domain by Plasmin Provides a New Mechanism for Regulating Cell Adhesion, J. Biol. Chem. 277 (2002) 45911-45919. doi:10.1074/jbc.M202637200.
[45] J.D. Hunter, Matplotlib: A 2D graphics environment, Comput. Sci. Eng. 9 (2007) 99-104. doi:10.1109/MCSE.2007.55.

1. A peptide comprising an amino acid sequence having at least 65% identity with SEQ ID No. 1 (FETLRGDLRILSILRHQNLLKELQD) or a functional fragment thereof said peptide or functional fragment thereof being in a linear form or in an intramolecular macrocyclic form.

2. The peptide or functional fragment thereof according to claim 1 being a ligand of integrins αvβ6 and αvβ8.

3. The peptide or functional fragment thereof according to claim 1 having a Ki for αvβ6 lower than 2 nM and/or a Ki for αvβ8 lower than 10 nM.

4. The peptide or functional fragment thereof according to claim 1, comprising FETLRGDLRILSIL (SEQ ID No. 2).

5. The peptide or functional fragment thereof according to claim 1 wherein the intramolecular macrocyclic form is obtained by a stapling method or is a head-to-tail cyclic form.

6. The peptide or functional fragment thereof according to claim 5 wherein the intramolecular macrocyclic form comprises a triazole-bridged macrocyclic scaffold.

7. The peptide or functional fragment thereof according to claim 6 wherein the triazole-bridged macrocyclic scaffold is present between residues in position 54 (propargylglycine) and 58 (azidolysine) or in position 54 (azidolysine) and 58 (propargylglycine).

8. The peptide or functional fragment thereof according to claim 6 ef-7 wherein the triazole-bridged macrocyclic scaffold is inserted through copper-catalyzed azide-alkyne cycloaddition.

9. The peptide or functional fragment thereof according to claim 5 wherein the intramolecular macrocyclic form has the structure of:

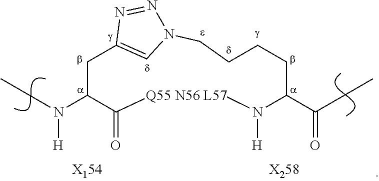

10. The peptide or functional fragment thereof according to claim 1, being fused with an agent, preferably said agent is an inorganic or organic nanoparticle, a therapeutic agent, a radioisotope, a chemotherapeutic agent, an antibody and/or an antibody fragment, a toxin, a nucleic acid, a RNA therapeutic agent, a diagnostic agent for radioimaging, fluorescence or photoacoustic imaging, a radioisotope fluorescent dye or a nanoparticle, a fluorescein, rhodamines, bodipys, indocyanines, porphyrines andphthalocyanines, IRDye ICG, methylene blue, omocyanine and quantum dots, a dye, a contrasting agents for MRI and Contrast-enhanced ultrasound (CEUS) or a cellular component.

11. A composition comprising the peptide or functional fragment thereof according to claim 1 and suitable carriers.

12. The composition according to claim 11 further comprising an agent, preferably said agent is an inorganic or organic nanoparticle, a therapeutic agent, a radioisotope, a chemotherapeutic agent, an antibody and/or an antibody fragment, a toxin, a nucleic acid, a RNA therapeutic agent, a diagnostic agent for radioimaging, fluorescence or photoacoustic imaging, a radioisotope fluorescent dye or a nanoparticle, a fluorescein, rhodamines, bodipys, indocyanines, porphyrines andphthalocyanines, IRDye ICG, methylene blue, omocyanine and quantum dots, a dye, a contrasting agents for MRI and Contrast-enhanced ultrasound (CEUS) or a cellular component.

13. (canceled)

14. The peptide or functional fragment thereof according to claim 1 for use in detecting a tumor, preferably the tumor overexpresses αvβ6 and αvβ8 integrins, preferably the tumor is oral or skin squamous cell carcinoma, head and neck, pancreatic, ovarian, lung, cervix, colorectal, gastric, prostatic and breast cancer, melanomas and brain tumors (e.g. glioblastoma and/or astrocytoma).

15. A method for the treatment of cancer or fibrosis, preferably the cancer overexpresses αvβ6 and αvβ8 integrins, preferably the cancer is oral or skin squamous cell carcinoma, head and neck, pancreatic, ovarian, lung, cervix, colorectal, gastric, prostatic and breast cancer, melanomas and brain tumors (e.g. glioblastoma and/or astrocytoma), comprising administering the peptide or functional fragment thereof according to claim 1 to a patient in need of such treatment.
